(19) United States
(12) Patent Application Publication
(10) Pub. No.: US 2024/0342164 A1
(43) Pub. Date: Oct. 17, 2024

(54) METHOD OF TREATMENT OF DEPRESSED PATIENTS WITH POOR COGNITION AND SELECTION OF OTHER PATIENTS BENEFITING FROM A BENZYLPIPERAZINE-AMINOPYRIDINE AGENT

(71) Applicant: Alto Neuroscience, Inc., Los Altos, CA (US)

(72) Inventors: Amit ETKIN, Los Altos, CA (US); Dan SEGAL, Los Altos, CA (US); Wei WU, Los Altos, CA (US); Li SHEN, Los Altos, CA (US); Vinit SHAH, Los Altos, CA (US); Adam SAVITZ, Los Altos, CA (US)

(21) Appl. No.: 18/670,580

(22) Filed: May 21, 2024

Related U.S. Application Data
(63) Continuation-in-part of application No. 17/805,374, filed on Jun. 3, 2022.
(60) Provisional application No. 63/202,254, filed on Jun. 3, 2021.
(60) Provisional application No. 63/263,168, filed on Oct. 28, 2021.
(60) Provisional application No. 63/264,269, filed on Nov. 18, 2021.

(51) Int. Cl. A61K 31/496; A61K 9/00; A61P 25/28

(52) U.S. Cl. CPC A61K 31/496; A61K 9/0053; A61P 25/28

(57) ABSTRACT

This invention relates to the use of (4-benzylpiperazin-1-yl)-[2-(3-methylbutylamino) pyridin-3-yl]methanone (NSI-189) or a pharmaceutically acceptable salt thereof in the treatment of a psychiatric condition in which depressive symptoms are prominent, including major depressive disorder, bipolar disorder, posttraumatic stress disorder, substance use disorder, and depression-related aspects of schizophrenia (e.g. negative symptoms), in a patient who is cognitively impaired or has poor or slow cognition or difficulty making decisions or exhibits certain EEG properties. The present invention also relates to the selection of patients with a biomarker (e.g., EEG) and/or clinical symptoms who would most benefit from such compounds.

This application is a continuation-in-part of U.S. patent application Ser. No. 17/805,374, filed Jun. 3, 2022, which claims the benefit of U.S. Provisional Application No. 63/202,254, filed Jun. 3, 2021, U.S. Provisional Application No. 63/263,168, filed Oct. 28, 2021, and U.S. Provisional Application No. 63/264,269, filed Nov. 18, 2021, all of which are hereby incorporated by reference in their entireties.

### FIELD OF THE INVENTION

This invention relates to the use of (4-benzylpiperazin-1-yl)-[2-(3-methylbutylamino)pyridin-3-yl]methanone (NSI-189) or a pharmaceutically acceptable salt thereof in the treatment of a psychiatric condition in which depressive symptoms are prominent, including major depressive disorder (MDD), bipolar disorder, posttraumatic stress disorder, substance use disorder, and depression-related aspects of schizophrenia (e.g. negative symptoms) in a patient, for example a patient who is cognitively impaired or has poor or slow cognition or difficulty making decisions or exhibits certain EEG properties. The present invention also relates to the selection of patients with a biomarker (e.g., EEG) and/or clinical symptoms who would most benefit from such compounds.

### BACKGROUND OF THE INVENTION

Clinical care for depression involves assessment and diagnosis based on a set of clinician-assessed and patient-reported symptoms such as depressed mood, anhedonia, diminished ability to think or concentrate, appetite changes, sleep and psychomotor changes but notably not based on biological or quantitative behavioral variables. When an assessment such as a magnetic resonance imaging (MRI) scan or a blood test is performed, it is to rule out non-psychiatric causes of depression which may necessitate treatments other than an antidepressant medication, including causes such as a tumor, hypothyroidism, dementia or metabolic disruptions. After diagnosing a patient with depression such as in major depressive disorder (MDD), a clinician may then prescribe one of multiple antidepressant treatments, which primarily includes drugs such as selective serotonin reuptake inhibitors (SSRIs), serotonin norepinephrine reuptake inhibitors (SNRIs), and norepinephrine dopamine reuptake inhibitors (NDRIs), or atypical antidepressants. Notably, however, selection of antidepressant medication is done purely by trial-and-error, with no symptom profile or biological or quantitative behavioral measures to inform medication choice. Typically, SSRIs are selected as the first line treatment based on their generally better tolerability, but not because they are known to be more effective generally, nor more effective for that particular patient. Most patients, however, fail to respond adequately to the first medication, at which point selection of the next medication again follows a trial-and-error process. Indeed, it has been found that on average, failing one SSRI does not necessarily predict a different response to another SSRI versus an SNRI or NDRI. Rush et al., N Engl J Med. 2006, 354:1231-1242. As such, typical clinical assessments do not provide information sufficiently useful for selection of subsequent medication trials, and therefore external information not available to the clinician is required for improving medication selection.

The economic, societal and personal cost of depression is very large, with depression being the leading cause of disability worldwide. This is even more pronounced for treatment-resistant depression (Amos et al., J Clin Psychiatry, 2018; 79), thus suggesting that finding the best medication for an individual early in the course of treatment would provide many downstream benefits to the patient and society at large.

Similar clinical needs exist in other related conditions in which depressive symptoms exist. Such symptoms include low mood, lack of experience of pleasure (anhedonia), impairments in motivation, and impairments in attention, cognition or decision making. These conditions include major depressive disorder, bipolar depression (such as bipolar I or bipolar II disorders), post-traumatic stress disorder, substance use disorder and depression-related aspects of schizophrenia (e.g., negative symptoms). Importantly, these different depression-related symptoms or areas of dysfunction co-occur and may be functionally related. For example, a patient with major depression may report depressed mood and lack of motivation. Likewise, the same symptoms may be reported by patients diagnosed with other conditions in which similar impairments may co-occur, such as bipolar depression, post-traumatic stress disorder or substance use disorder. Though schizophrenia is often thought of with respect to prominent hallucinations and delusions, the depression-like negative symptoms are often the greater source of long-term disability and functional impairment. Hence, a treatment approach that encompasses these multiple and related functional systems would be both of importance to any one of these clinical conditions, and equally may be applicable across them.

It has been proposed that depression and depressive symptoms arise at least in part from impairments in neuronal proliferation in the adult brain, neuronal growth and differentiation, elaboration of neuronal substructures important in neural communication, and impairments in neural plasticity (1-7). Moreover, it has been theorized that medications that reverse one or multiple of these dysfunctions would be effective antidepressants (1-7). It has been demonstrated, for example, that medications that have been clinically proven to be effective antidepressants in humans with depression also induce the growth and differentiation of neurons in the brains of adult animals (1, 3, 4).

Prior work has found that certain benzylpiperazine-aminopyridines or open chain forms thereof can induce proliferation and maturation of neurons in the adult brains of animals (8, 9). One such benzylpiperazine-aminopyridine, NSI-189, was discovered by screening a chemical library against an in vitro model for hippocampal neurogenesis. Unlike most recently FDA approved antidepressants (such as selective serotonin reuptake inhibitors, SSRIs), the precise mechanism of action of NSI-189 is not understood. There are no members of its class that have received FDA approval. An initial small-scale Phase 1b clinical study in patients with major depression found promising evidence for potential antidepressant efficacy of NSI-189 dosed at total daily doses of 40 mg, 80 mg or 120 mg (10). However, this study was conducted on a small sample of patients and had as its goal to establish safety and tolerability, and thus did not yield definitive efficacy data, with only 18 patients across all active dose condition (and 6 on placebo).

A subsequent Phase 2 study was then performed on 220 patients with major depression (11). In that study, patients were randomized to receive a total daily dose of 40 mg or 80 mg of NSI-189 or placebo in stage 1 of the clinical trial design. Patients receiving placebo and who failed to respond to placebo were then re-randomized to 40 mg, 80 mg or placebo in stage 2 of the trial. Treatment in either stage was given for six weeks. This design is termed a Sequential Parallel Clinical Design (SPCD), and is analyzed statistically by combining results in a pre-defined manner for outcomes differences between relevant groups (e.g., drug vs. placebo) in each of the stages individually. There are two depression outcome measures accepted by the United States Food and Drug Administration (FDA) for the evaluation of adults. See the June 2018 FDA draft guidance entitled “Major Depressive Disorder: Developing Drugs for Treatment.” These are the Montgomery-Asberg Depression Rating Scale (MADRS), which was the primary outcome in this study, as well as the Hamilton Depression Rating Scale (HDRS) which was a key secondary outcome measure. However, despite the promise of animal work and early human studies, results from this 220-patient study failed to demonstrate any evidence of antidepressant efficacy on the MADRS primary outcome measure or the HDRS key secondary measure (11) across either the full SPCD analysis or analyses of each stage alone. The 80 mg daily dose did not demonstrate efficacy on any study measure (11). It was reported that any benefits of NSI-189 on depression and cognition are independent effects (27).

While nominally significant effects were observed for the 40 mg dose compared to placebo (but not the 80 mg dose) on several secondary measures, these results were not corrected for multiple comparisons (nor would have survived correction) and do not reflect measures acceptable to the US FDA in support of antidepressant efficacy for a medication. Thus, NSI-189 was not found to be an effective antidepressant. Moreover, inasmuch as uncorrected results on secondary measures can even be used to draw positive conclusions regarding efficacy of the compound, these point to potential efficacy for 40 mg and not 80 mg. This would be surprising as it would reflect an inverted dose-response curve, while the typical expectation is that a higher dose would be more effective than a lower dose. Together, these results led to discontinuance of the development of NSI-189.

There is a continuing need for improved treatments for depression, such as antidepressants which have a different mechanism of action than those currently used for treatment.

### SUMMARY OF THE INVENTION

The inventors discovered that 80 mg of (4-benzylpiperazin-1-yl)-[2-(3-methylbutylamino)pyridin-3-yl]methanone (NSI-189) is effective in treating depression in objectively determined-cognitively poor, slow or impaired patients or those patients with difficulty making decisions. This is particularly surprising since NSI-189 was previously reported to be ineffective in treating major depressive disorder, and that the 80 mg dose did not demonstrate efficacy on any study measure for the entire study population (including all secondary measures and even when not correcting for multiple comparisons).

One embodiment is a method of treating major depressive disorder in a human patient having objectively determined-cognitive impairment, poor or slow cognition or a patient with difficulty making decisions comprising administering (e.g., orally) to the patient an effective amount of NSI-189 or a pharmaceutically acceptable salt thereof (e.g., orally administering from about 40 to about 120, 160, or 240 mg (such as about 40 to about 120 mg, or about 60 mg to about 100 mg) of NSI-189 or a pharmaceutically acceptable salt thereof daily). In a preferred embodiment, about 80 mg of NSI-189 or a pharmaceutically acceptable salt thereof (such as NSI-189 phosphate) is orally administered daily. The cognitive impairment, poor or slow cognition or difficulties making decisions of the patient may be diagnosed by one or more of a simple reaction time test, choice reaction time test, one back working memory task, and visual learning task. In one embodiment, the patient also suffers from anhedonia, suicidality, or both. In another embodiment, the patient is not concurrently treated with an antidepressant medication other than NSI-189. In yet another embodiment, the patient is concurrently treated with one or more antidepressants (other than NSI-189 or a pharmaceutically acceptable salt thereof). In yet another embodiment, the patient, prior to treatment with NSI-189, is treated with an antidepressant (other than NSI-189 or a pharmaceutically acceptable salt thereof) but nonetheless continues to have depressive symptoms.

Another embodiment is a method for treating objectively determined-cognitive impairment, poor or slow cognition, difficulty making decisions, or reduced information processing speed in a human patient suffering from major depressive disorder comprising administering to the patient an effective amount of NSI-189 or a pharmaceutically acceptable salt thereof (e.g., oral administering from about 40 to about 120, 160, or 240 mg (such as about 40 to about 120 mg, or about 60 mg to about 100 mg) of NSI-189 or a pharmaceutically acceptable salt thereof daily). In a preferred embodiment, about 80 mg of NSI-189 or a pharmaceutically acceptable salt thereof (such as NSI-189 phosphate) is administered daily.

Yet another embodiment is a method of treating major depressive disorder in a human patient having reduced information processing speed, slow decision making or difficulty making decisions comprising administering to the patient an effective amount of NSI-189 or a pharmaceutically acceptable salt thereof (e.g., oral administering from about 40 to about 120, 160, or 240 mg (such as about 40 to about 120 mg, or about 60 mg to about 100 mg) of NSI-189 or a pharmaceutically acceptable salt thereof daily). In a preferred embodiment, about 80 mg of NSI-189 or a pharmaceutically acceptable salt thereof (such as NSI-189 phosphate) is orally administered daily. In one embodiment, the patient also suffers from anhedonia, suicidality, or both. In another embodiment, the patient is not concurrently treated with an antidepressant medication other than NSI-189. In yet another embodiment, the patient is concurrently treated with one or more antidepressants (other than NSI-189 or a pharmaceutically acceptable salt thereof).

Yet another embodiment is a method of treating major depressive disorder in a human patient having reduced attention, memory, learning, information processing speed, working memory, or any combination of any of the foregoing comprising administering to the patient an effective amount of NSI-189 or a pharmaceutically acceptable salt thereof (e.g., oral administering from about 40 mg to about 120, 160, or 240 mg, such as about 40 to about 120 mg or about 60 mg to about 100 mg of NSI-189 or a pharmaceutically acceptable salt thereof daily). In a preferred embodiment, about 80 mg of NSI-189 or a pharmaceutically acceptable salt thereof (such as NSI-189 phosphate) is orally administered daily. The reduced attention, memory, learning, information processing speed or working memory may be diagnosed by tests known in the art such as the Digit symbol substitution task, Oddball task, Flanker task, Wisconsin card sort task, Trail making task, Corsi Block task, Digit Span task, Reverse Digit Span task, Verbal Learning and Memory task, Verbal Fluency task, symbol digit modalities test, Wechsler Adult Intelligence Scale (WAIS) coding subtest, digit vigilance test, d2 test of attention, WAIS symbol search subtest, WAIS cancellation subtest, Neuropsychological Assessment Battery (NAB) Numbers and letters subtest, Ruff 2&7 selective attention test, Stroop Color/Word test, NAB mazes and other maze tests, Delis-Kaplan Executive Function System (D-KEFS) design fluency subtest and Repeatable Battery for the Assessment of Neuropsychological Status (RBANS) coding subtest. Measurements in these tasks include reaction time, accuracy and variation in reaction times. For learning tasks, measurements additionally include learning rate, items recalled, items recognized and interference effects from distractor lists. In one embodiment, the patient also suffers from anhedonia, suicidality, or both. In another embodiment, the patient is not concurrently treated with an antidepressant medication other than NSI-189. In yet another embodiment, the patient is concurrently treated with one or more antidepressants (other than NSI-189 or a pharmaceutically acceptable salt thereof).

Yet another embodiment is a method of assessing and treating major depressive disorder in a human patient comprising:
(a) objectively assessing whether a patient having major depressive disorder is cognitively impaired, suffers from poor or slow cognition, from difficulty making decisions, or reduced information processing speed;
(b) upon an assessment from step (a) that the patient is cognitively impaired or has poor cognition, initiating oral administration to the patient of from about 40 to about 120, 160, or 240 mg (such as about 40 to about 120 mg, from about 60 mg to about 100 mg, or about 80 mg) of (4-benzylpiperazin-1-yl)-[2-(3-methylbutylamino)pyridin-3-yl]methanone or a pharmaceutically acceptable salt thereof daily; and
(c) upon an assessment from step (a) that the patient is not cognitively impaired and does not have poor cognition, not initiating treatment with (4-benzylpiperazin-1-yl)-[2-(3-methylbutylamino)pyridin-3-yl]methanone or a pharmaceutically acceptable salt. In one embodiment, the patient is concurrently treated with one or more antidepressants (other than NSI-189 or a pharmaceutically acceptable salt thereof).

Yet another embodiment is a method of assessing and treating major depressive disorder in a human patient comprising:
(a) assessing whether a patient having major depressive disorder has reduced information processing speed, attention, memory, learning, or working memory, slow decision making, difficulty making decisions, or any combination of any of the foregoing; and
(b) upon an assessment from step (a) that the patient has reduced information processing speed, attention, memory, learning, or working memory, slow decision making, difficulty making decisions, or any combination of any of the foregoing, initiating oral administration to the patient of from about 40 to about 120, 160, or 240 mg (such as about 40 to about 120 mg, from about 60 mg to about 100 mg, or about 80 mg) of (4-benzylpiperazin-1-yl)-[2-(3-methylbutylamino)pyridin-3-yl]methanone or a pharmaceutically acceptable salt thereof daily. In one embodiment, the patient is concurrently treated with one or more antidepressants (other than NSI-189 or a pharmaceutically acceptable salt thereof).

Yet another embodiment is a method of treating late-life depression in a human patient at least 50 years of age comprising administering to the patient an effective amount of NSI-189 or a pharmaceutically acceptable salt thereof (e.g., oral administering from about 40 to about 120, 160, or 240 mg (such as about 40 to about 120 mg, about 60 mg to about 100 mg, or about 80 mg) of NSI-189 or a pharmaceutically acceptable salt thereof daily). In a preferred embodiment, about 80 mg of NSI-189 or a pharmaceutically acceptable salt thereof (such as NSI-189 phosphate) is administered daily. In one embodiment, the patient is at least 60 or 65 years of age. In another embodiment, the patient is not concurrently treated with an antidepressant medication (other than NSI-189). In yet another embodiment, the patient is concurrently treated with one or more antidepressants (other than NSI-189 or a pharmaceutically acceptable salt thereof). In one embodiment the patient has late life onset depression.

Yet another embodiment is a method of treating one or more symptoms including depressive symptoms, anhedonia, loss of interest, avolition, diminished emotional expression, inability to feel, amotivation, apathy, slow thinking, psychomotor retardation, lassitude, or any combination of any of the foregoing, in a human patient suffering from post-traumatic stress disorder (PTSD), bipolar depression, substance use disorder or schizophrenia comprising administering to the patient an effective amount of NSI-189 or a pharmaceutically acceptable salt thereof (e.g., oral administering from about 40 to about 120, 160, or 240 mg, such as about 40 to about 120 mg, from about 60 mg to about 100 mg, or about 80 mg of NSI-189 or a pharmaceutically acceptable salt thereof daily). In a preferred embodiment, about 80 mg of NSI-189 or a pharmaceutically acceptable salt thereof (such as NSI-189 phosphate) is administered daily. In one embodiment, the patient suffers from cognitive impairment, poor or slow cognition, difficulty making decisions, or reduced information processing speed. In another embodiment, the patient is not concurrently treated with an antidepressant medication (other than NSI-189). In yet another embodiment, the patient is concurrently treated with one or more antidepressants (other than NSI-189 or a pharmaceutically acceptable salt thereof). In yet another embodiment, the patient is concurrently treated with one or more antipsychotic medications, mood stabilizers, or any combination of any of the foregoing.

Yet another embodiment is a method of treating negative and/or cognitive symptoms of schizophrenia in a human patient comprising administering to the patient an effective amount of NSI-189 or a pharmaceutically acceptable salt thereof (e.g., oral administering from about 40 to about 120, 160, or 240 mg of NSI-189 or a pharmaceutically acceptable salt thereof daily). In a preferred embodiment, about 80 mg of NSI-189 or a pharmaceutically acceptable salt thereof (such as NSI-189 phosphate) is administered daily. In one embodiment, the patient is treated for negative symptoms of schizophrenia. In another embodiment, the patient is treated for cognitive symptoms of schizophrenia. In yet another embodiment, the patient is treated for negative symptoms and cognitive symptoms of schizophrenia. In one embodiment, the patient suffers from cognitive impairment, poor or slow cognition, difficulty with decision making, or reduced information processing speed. In another embodiment, the patient is not concurrently treated with one or more antipsychotic medications. In yet another embodiment, the patient is concurrently treated with one or more antipsychotic medications, mood stabilizers, or any combination of any of the foregoing. In another embodiment, the patient is not concurrently treated with an antidepressant medication (other than NSI-189). In yet another embodiment, the patient is concurrently treated with one or more antidepressants (other than NSI-189 or a pharmaceutically acceptable salt thereof).

It has also been found that the effects of NSI-189 or a pharmaceutically acceptable salt thereof can be predicted by behavioral or electroencephalographic (EEG) measures. These measures identify patients who benefit from such compounds significantly more than placebo. For instance, it has been found that patients exhibiting low power at the centro-parietal electrodes in the theta frequencies, low power at the centro-parietal electrodes in the alpha frequencies, low power at the frontal electrodes in the alpha frequencies, and high aperiodic exponent at one or more posterior electrodes are responsive to treatment with NSI-189. The present invention therefore includes the use of these measures as a method by which to identify those patients who would most benefit from treatment with NSI-189. In one embodiment of any of the methods described herein, the EEG recording of the patient exhibits low power at the centro-parietal electrodes in the theta frequencies, low power at the centro-parietal electrodes in the alpha frequencies, low power at the frontal electrodes in the alpha frequencies, high aperiodic exponent at one or more posterior electrodes, or any combination of any of the foregoing.

One embodiment is a method of treating major depressive disorder, bipolar disorder, late-life depression, schizophrenia, posttraumatic stress disorder, substance use disorder, depressive symptoms or negative symptoms in a patient comprising:
(a) receiving data comprised of one or more neurophysiological measures of the patient;
(b) optionally, receiving data comprised of one or more indicators of cognitive impairment, slow or poor cognition or difficulty making decisions in the patient; and
(c) administering to the patient an effective amount of NSI-189 or a pharmaceutically acceptable salt thereof, where the patient is determined to be responsive to NSI-189 based on the data comprised of the one or more neurophysiological measures and optionally the one or more indicators of cognitive impairment, slow or poor cognition or difficulty making decisions. In one embodiment, the patient suffers from bipolar disorder, late-life depression, schizophrenia, posttraumatic stress disorder, or substance use disorder in which depressive symptoms are prominent. In one embodiment, the patient is concurrently treated with one or more antidepressants (other than NSI-189 or a pharmaceutically acceptable salt thereof), mood stabilizers, or any combination of any of the foregoing. In another embodiment, the patient is not concurrently treated with an antidepressant medication (other than NSI-189).

The neurophysiological measure can be a measure of brain activity, such as with electroencephalogram (EEG) recordings. The electroencephalogram (EEG) recordings can measure power of one or more frequencies, power ratios between frequencies, cordance, power envelope connectivity, coherence, imaginary coherence, phase locking value, phase lag index, weighted phase lag index, covariance, cross-frequency coupling, aperiodic exponent, alpha peak frequency, alpha peak frequency proximity, or information theoretical indices and entropy. In one embodiment, the EEG recording of the patient exhibits low power at the centro-parietal electrodes in the theta frequencies, low power at the centro-parietal electrodes in the alpha frequencies, low power at the frontal electrodes in the alpha frequencies, high aperiodic exponent at one or more posterior electrodes, or any combination of any of the foregoing.

The one or more indicators of cognitive impairment, slow or poor cognition or difficulty making decisions can include, e.g., one or more measurements from a simple reaction time task, a choice reaction time task, a one-back working memory task, and a visual learning task, and a self-report questionnaire. Other indicators include, e.g., one or more measurements from a Digit symbol substitution task, Oddball task, Flanker task, Wisconsin card sort task, Trail making task, Corsi Block task, Digit Span task, Reverse Digit Span task, Verbal Learning and Memory task, Verbal Fluency task, symbol digit modalities test, Wechsler Adult Intelligence Scale (WAIS) coding subtest, digit vigilance test, d2 test of attention, WAIS symbol search subtest, WAIS cancellation subtest, Neuropsychological Assessment Battery (NAB) Numbers and letters subtest, Ruff 2&7 selective attention test, Stroop Color/Word test, NAB mazes and other maze tests, Delis-Kaplan Executive Function System (D-KEFS) design fluency subtest, or Repeatable Battery for the Assessment of Neuropsychological Status (RBANS) coding subtest. The one or more indicators of cognitive impairment can be calculated as z-scores normalizing the patient against a healthy population. In one embodiment, the one or more indicators of cognitive impairment, slow or poor cognition or difficulty making decisions are merged into a composite cognitive task performance score.

In one embodiment, machine learning or multivariate modeling is applied to predict the responsiveness of the patient to the administration of NSI-189.

In one embodiment of any of the methods described herein, from about 40 to about 120, 160, or 240 mg/day of NSI-189 or a pharmaceutically acceptable salt thereof is administered to the patient. For instance, from about 40 to about 120 mg, from about 60 to about 120 mg/day, from about 70 to about 120 mg/day, or from about 80 to about 100 mg/day of NSI-189 or a pharmaceutically acceptable salt thereof is administered to the patient. In a preferred embodiment, about 80 mg/day of NSI-189 or a pharmaceutically acceptable salt thereof is administered to the patient. In any of the methods described herein, the preferred administration route is orally.

In one embodiment of any of the methods described herein, NSI-189 or a pharmaceutically acceptable salt thereof is administered daily such that, at steady state (such as 28 days after initiating treatment with NSI-189), the Cₘₐₓ for NSI-189 is about 230 to about 630 ng/ml (e.g., about 300 to about 550 ng/ml). In another embodiment, NSI-189 or a pharmaceutically acceptable salt thereof is administered daily such that, at steady state (such as 28 days after initiating treatment with NSI-189), the AUC₀₋₂₄ for NSI-189 is about 1400 to about 3200 hr·ng/ml (e.g., about 1500 to about 3100 hr·ng/mL, about 1600 to about 3000 hr·ng/ml, about 1700 to about 2900 hr·ng/mL, or about 1800 to about 2800 hr·ng/mL). In yet another embodiment, NSI-189 or a pharmaceutically acceptable salt thereof is administered daily such that, at steady state (such as 28 days after initiating treatment with NSI-189), the AUC₀₋ₜₐᵤ for NSI-189 is about 1200 to about 2050 hr·ng/ml (e.g., about 1400 to about 1850 hr·ng/ml).

In one embodiment of any of the methods described herein, the method comprises orally administering 40 mg of NSI-189 or a pharmaceutically acceptable salt thereof twice daily (b.i.d.). In one embodiment, the 40 mg b.i.d. administration provides, at steady state (such as 28 days after initiating treatment with NSI-189), a Cₘₐₓ for NSI-189 of about 230 to about 630 ng/ml (e.g., about 300 to about 550 ng/ml). In another embodiment, the 40 mg b.i.d. administration provides, at steady state (such as 28 days after initiating treatment with NSI-189), an AUC₀₋₂₄ for NSI-189 of about 1400 to about 3200 hr·ng/ml (e.g., about 1500 to about 3100 hr·ng/ml, about 1600 to about 3000 hr·ng/ml, about 1700 to about 2900 hr·ng/mL, or about 1800 to about 2800 hr·ng/mL). In yet another embodiment, the 40 mg b.i.d. administration provides, at steady state (such as 28 days after initiating treatment with NSI-189), an AUC₀₋ₜₐᵤ for NSI-189 of about 1200 to about 2050 hr·ng/ml (e.g., about 1400 to about 1850 hr·ng/ml).

Another embodiment is a method of treating (i) major depressive disorder, (ii) late-life depression, or (iii) depressive symptoms associated with PTSD, bipolar depression, substance use disorder or schizophrenia in a human patient diagnosed based on EEG signals for parietal and/or frontal electrodes (e.g., centro-parietal electrodes) in the theta and/or alpha frequencies by administering to the patient an effective amount of NSI-189 or a pharmaceutically acceptable salt thereof (e.g., orally administering from about 40 to about 160 mg, about 40 to about 120 mg, about 60 mg to about 100 mg (e.g., 80 mg) of NSI-189 or a pharmaceutically acceptable salt thereof). In one embodiment, the patient to be administered NSI-189 or a pharmaceutically acceptable salt thereof exhibits low power (e.g., below average power) in parietal and/or frontal electrodes (e.g., centro-parietal electrodes) in the theta and/or alpha frequencies. In another embodiment, the patient to be administered NSI-189 or a pharmaceutically acceptable salt thereof exhibits a high aperiodic exponent (e.g., above average aperiodic exponent) in occipital, parietal and/or temporal electrodes. In another embodiment, the patient is concurrently treated with one or more antidepressants (other than NSI-189 or a pharmaceutically acceptable salt thereof). In one embodiment, the patient is concurrently treated with one or more mood stabilizers.

Yet another embodiment is a method of treating a patient suffering from a substance use disorder and cognitive impairment, slow or poor cognition or difficulty making decisions by administering to the patient an effective amount of NSI-189 or a pharmaceutically acceptable salt thereof (e.g., orally administering from about 40 mg to about 120, 160, or 240 mg, such as about 40 to about 120 mg, or about 60 to about 100 mg (e.g., 80 mg) of NSI-189 or a pharmaceutically acceptable salt thereof).

Yet another embodiment is a method of treating sadness, low mood, inability to feel, anhedonia, psychomotor retardation, lassitude, suicidality, guilt or concentration difficulties in a human patient suffering from major depressive disorder by administering to the patient an effective amount of NSI-189 or a pharmaceutically acceptable salt thereof (e.g., orally administering from about 40 to about 120, 160, or 240 mg, such as about 40 to about 120 mg, or about 60 mg to about 100 mg (e.g., 80 mg) of NSI-189 or a pharmaceutically acceptable salt thereof). In another embodiment, the patient is concurrently treated with one or more antidepressants (other than NSI-189 or a pharmaceutically acceptable salt thereof).

In one embodiment of any of the aforementioned methods, the patient has previously been treated with one or more antidepressants but failed to respond to them, and continues to be treated with the one or more antidepressants even after NSI-189 (or a pharmaceutically acceptable salt thereof) treatment is begun. In other words, the NSI-189 or a pharmaceutically acceptable salt thereof is provided as an adjunctive therapy to the one or more antidepressants. In one embodiment, the one or more antidepressants do not include a monoamine oxidase inhibitor (MAOI) or a tricyclic antidepressant. In another embodiment, the one or more antidepressants are selected from serotonin reuptake inhibitors, serotonin and norepinephrine reuptake inhibitors, mirtazapine, bupropion, and any combination of any of the foregoing. The patient may suffer from major depressive disorder and/or posttraumatic disorder. In one embodiment, 40 mg of the NSI-189 or a pharmaceutically acceptable salt thereof, such as NSI-189 phosphate, is orally administered twice daily.

In one embodiment, the patient suffers from anhedonia. In another embodiment, the patient suffers from suicidality.

Yet another embodiment is a system comprising:
at least one data processor; and
at least one memory storing instructions which, when executed by the at least one data processor, result in operations comprising:
(a) receiving data comprised of one or more neurophysiological measures in a patient;
(b) optionally, receiving data comprised of one or more indicators of cognitive impairment or poor cognition in the patient;
(c) analyzing, using a multivariate model (e.g., a machine learning model), (i) the data comprised of one or more neurophysiological measures in the patient and (ii) optionally the data comprised of one or more indicators of cognitive impairment or poor cognition, to predict the responsiveness of the patient to NSI-189; and
(d) outputting a prediction of the responsiveness of the patient to NSI-189 based on the analyzed data.

Prior to step (c), the multivariate model (such as machine learning model) may be used to analyze data from prior patients receiving NSI-189 (or a pharmaceutically acceptable salt thereof) and one or more of their neurophysiological measures and optionally one of more of their indicators of cognitive impairment.

In one embodiment, the instructions result in operations further comprising outputting a recommendation to administer an effective amount of NSI-189 or a pharmaceutically acceptable salt thereof.

The neurophysiological measure can be electroencephalogram (EEG) recordings, such as EEG recordings. The electroencephalogram (EEG) recordings can measure power of one or more frequencies, power ratios between frequencies, cordance, power envelope connectivity, coherence, imaginary coherence, phase locking value, phase lag index, weighted phase lag index, covariance, cross-frequency coupling, aperiodic exponent, alpha peak frequency, alpha peak frequency coherence, or information theoretical indices and entropy. In one embodiment, the EEG recording of the patient exhibits low power at the centro-parietal electrodes in the theta frequencies, low power at the centro-parietal electrodes in the alpha frequencies, low power at the frontal electrodes in the alpha frequencies, high aperiodic exponent at one or more posterior electrodes, or any combination of any of the foregoing.

The one or more indicators of cognitive impairment, slow or poor cognition or difficulty making decisions can include one or more measurements from a simple reaction time task, a choice reaction time task, a one-back working memory task, and a visual learning task, and a self-report questionnaire. Other indicators include one or more measurements from a Digit symbol substitution task, Oddball task, Flanker task, Wisconsin card sort task, Trail making task, Corsi Block task, Digit Span task, Reverse Digit Span task, Verbal Learning and Memory task, Verbal Fluency task, symbol digit modalities test, Wechsler Adult Intelligence Scale (WAIS) coding subtest, digit vigilance test, d2 test of attention, WAIS symbol search subtest, WAIS cancellation subtest, Neuropsychological Assessment Battery (NAB) Numbers and letters subtest, Ruff 2&7 selective attention test, Stroop Color/Word test, NAB mazes and other maze tests, Delis-Kaplan Executive Function System (D-KEFS) design fluency subtest, or Repeatable Battery for the Assessment of Neuropsychological Status (RBANS) coding subtest. The one or more indicators of cognitive impairment, slow or poor cognition or difficulty making decisions can be calculated as z-scores normalizing the patient against a healthy population. These z-scores may be calculated based on measurements such as reaction time, accuracy and variation in reaction time. In one embodiment, the one or more indicators of cognitive impairment, slow or poor cognition or difficulty making decisions are merged into a composite cognitive task performance score.

In one embodiment, machine learning or multivariate modeling is applied to predict the responsiveness of the patient to the administration of NSI-189.

In one embodiment, from about 10 to about 130 mg/day of NSI-189 or a pharmaceutically acceptable salt thereof is administered to the patient. For instance, from about 20 to about 120 mg/day, from about 80 to about 120 mg/day, or from about 10 to about 40 mg/day of NSI-189 or a pharmaceutically acceptable salt thereof is administered to the patient. In a preferred embodiment, about 80 mg/day of NSI-189 or a pharmaceutically acceptable salt thereof is administered to the patient.

### BRIEF DESCRIPTION OF THE DRAWINGS

For a more complete understanding of the present invention, including features and advantages, reference is now made to the detailed description of the invention along with the accompanying figures.

FIG. 1A is a graph showing the MADRS depression score during stage 1 and stage 2 in all patients (i.e., all-comers analysis).

FIG. 1B is a graph showing the MADRS depression score during stage 1 and stage 2 of the study in poor cognition patients (as defined by the cognitive composite score being below the patient mean) receiving placebo or 40 or 80 mg NSI-189.

FIG. 1C is a graph showing the MADRS depression score during stage 1 and stage 2 of the study in good cognition patients (as defined by the cognitive composite score being above the patient mean) receiving placebo or 40 or 80 mg NSI-189.

FIG. 2A are graphs showing the MADRS depression score during stage 1 and stage 2 of the study in poor cognition patients (as defined by the choice RT task performance being below the patient mean).

FIG. 2B are graphs showing the MADRS depression score during stage 1 and stage 2 of the study in good cognition patients (as defined by the choice RT task performance being above the patient mean).

FIG. 2C are graphs showing the MADRS depression score during stage 1 and stage 2 of the study in poor cognition patients (as defined by the simple RT task performance being below the patient mean).

FIG. 2D are graphs showing the MADRS depression score during stage 1 and stage 2 of the study in good cognition patients (as defined by the simple RT task performance being above the patient mean).

FIG. 2E are graphs showing the MADRS depression score during stage 1 and stage 2 of the study in poor cognition patients (as defined by the working memory task performance being below the patient mean).

FIG. 2F are graphs showing the MADRS depression score during stage 1 and stage 2 of the study in good cognition patients (as defined by the working memory task performance being above the patient mean).

FIG. 2G are graphs showing the MADRS depression score during stage 1 and stage 2 of the study in poor cognition patients (as defined by the visual learning task performance being below the patient mean).

FIG. 2H are graphs showing the MADRS depression score during stage 1 and stage 2 of the study in good cognition patients (as defined by the visual learning task performance being above the patient mean).

FIG. 3 is a graph showing the MADRS depression score in poor cognition versus good cognition patients (as defined by the composite score being below or above the patient mean, respectively) in the 80 mg treatment arm from stage 1, over all 12 weeks of treatment. These patients continued on NSI-189 during stage 2 of the trial, allowing examination of the durability of the treatment benefit of the drug in cognitively impaired patients.

FIG. 4 is a bar graph showing remission rates (as determined by the MADRS depression score) for all comers and poor cognition patients (based on the composite cognitive task performance score) for stage 1 after 6 weeks of treatment.

FIG. 5A are graphs showing the HDRS depression score during stage 1 and stage 2 of the study in poor cognition patients, as defined by the composite cognitive task performance score being below the patient mean.

FIG. 5B are graphs showing the HDRS depression score during stage 1 and stage 2 of the study in good cognition patients, as defined by the composite cognitive task performance score being above the patient mean.

FIG. 6A are graphs showing the CGI severity during stage 1 and stage 2 of the study in poor cognition patients, as defined by the composite cognitive task performance score being below the patient mean.

FIG. 6B are graphs showing the CGI severity during stage 1 and stage 2 of the study in good cognition patients, as defined by the composite cognitive task performance score being above the patient mean.

FIG. 7 are tables showing the effect size (using Cohen's d) for all comers and for poor cognition patients (as defined either by the cognitive composite score or choice RT task being below the patient mean) on the MADRS for stage 1 and stage 2 of the treatment study, shown for the 6 week time point. Effect sizes reflect the difference between the 80 mg and placebo arms.

FIG. 8A is a graph showing the MADRS depression score in cognitively impaired patients as defined by the cognitive composite score being below the patient mean.

FIG. 8B is a graph showing the MADRS depression score in cognitively impaired patients as defined by a CPFQ score >25.

FIG. 9A is a graph showing the MADRS depression score in late life depression patients (age 50 or greater).

FIG. 9B is a graph showing the MADRS depression score in non-late life depression patients (age <50).

FIG. 10 are tables showing MADRS item effect size during stage 1 and stage 2 of the study with respect to change from pre-treatment to six weeks of treatment in the 80 mg group compared to placebo, expressed as Cohen's d effect size measures. Positive values denote greater symptom reduction in the 80 mg group relative to placebo.

FIG. 11 shows performance of healthy individuals, moderate to severe depression patients with slow cognition and moderate to severe depression patients with non-slow cognition (defined based on a clustering analysis) in multiple cognitive tests from a battery that included the following tasks: simple reaction time (RT) task, choice reaction time task, Eriksen flanker task, digit symbol substitution task (DSST), Corsi block task, verbal learning and memory task, a typing-adapted version of a verbal fluency, trail making test, Wisconsin card sorting task (WCST), delay discounting task, effortful expenditure for reward task and the facial emotion recognition test. All scores are plotted as z-scores after regressing out age and gender to ensure that these did not confound the results. Statistical tests reflect the comparison of the slow and non-slow patient groups.

FIG. 12A shows images of EEGs that predict treatment outcome. Shown are correlations between resting EEG power in the eyes closed condition and pre-minus post-treatment change in MADRS depression scores with treatment. In each pair of images, the lefthand image shows locations that are positively or negatively correlated with treatment outcome, plotting correlation coefficients. The righthand image shows electrodes and their significance (thresholded at p<0.05). The top set of images are for correlations amongst patients receiving NSI-189 (“NSI”) and the bottom set of images are for correlations amongst patients receiving placebo (“PBO”). The strongest predictive signals were for centro-parietal electrodes in the theta and alpha frequencies, as well as frontal electrodes in the alpha frequency, wherein lower power was predictive of better treatment outcome with the drug (evident by a negative correlation). For patients treated with placebo correlations were weaker and slightly positive (i.e. weakly in the opposite direction from drug).

FIG. 12B shows images of EEGs that predict treatment outcome. Specifically, the images show correlations between the aperiodic exponent in the eyes open and eyes closed conditions and pre minus post treatment change in MADRS depression scores with treatment. (A description of the aperiodic exponent is provided in, and illustrated at FIGS. 1A and 1B of, A. T. Hill et al., Dev. Cogn. Neurosci. 54:101076, 2022.) In each pair of images, the lefthand image shows locations that are positively or negatively correlated with treatment outcome, plotting correlation coefficients. The righthand image shows electrodes and their significance (thresholded at p<0.05). The strongest predictive signals were for posterior electrodes (occipital, parietal and temporal), wherein a higher aperiodic exponent was predictive of better treatment outcome with the drug (evident by a positive correlation). For patients treated with placebo correlations were weaker and negative (i.e. in the opposite direction from drug).

### DETAILED DESCRIPTION OF THE INVENTION

Unless specifically stated or obvious from context, as used herein, the term “or” is understood to be inclusive.

Unless specifically stated or obvious from context, as used herein, the terms “a”, “an”, and “the” are understood to be singular or plural.

Ranges provided herein are understood to be shorthand for all of the values within the range.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein can be modified by the term about.

The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

Unless indicated otherwise, the term “NSI-189” refers to (4-benzylpiperazin-1-yl)-[2-(3-methylbutylamino)pyridin-3-yl]methanone, which has the structure:

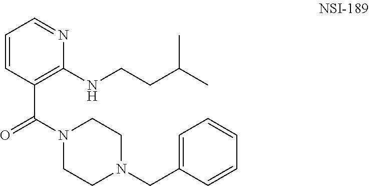 [SMILES: CC(C)CCNc1ncccc1C(=O)N1CCN(Cc2ccccc2)CC1]

NSI-189 can be synthesized as described in U.S. Pat. Nos. 7,560,553, 7,858,628, 9,278,933, and 9,572,807, each of which is incorporated by reference in its entirety. Pharmaceutically acceptable salts of NSI-189 include, but are not limited to, halides, maleates, succinates, nitrates, and phosphates. A preferred pharmaceutically acceptable salt of NSI-189 is (4-benzylpiperazin-1-yl)-[2-(3-methylbutylamino)pyridin-3-yl]methanone phosphate (such as (4-benzylpiperazin-1-yl)-[2-(3-methylbutylamino)pyridin-3-yl]methanone monophosphate also referred to as NSI-189 phosphate). NSI-189 or its pharmaceutically acceptable salt can be administered in the form of a dosage form containing one or more pharmaceutically acceptable excipients, such as an oral dosage form (e.g., a tablet, capsule, granules, or oral liquid).

The terms major depressive disorder, bipolar disorder, bipolar I disorder, bipolar II disorder, posttraumatic stress disorder, substance use disorder and schizophrenia are intended to be as defined in the Diagnostic and Statistical Manual of Mental Disorders, 5^{th} Ed. (“DSM-5”), American Psychiatric Association, 2013, which is hereby incorporated by reference.

As used herein, the term “brain activity” or “brain activity levels” refer to measurable (e.g., quantifiable) neural activity. Measurable neural activity includes, but is not limited to, a magnitude of activity, a frequency of activity, a delay of activity, or a duration of activity. Brain activity levels may be measured (e.g., quantified) during periods in which no stimulus is presented. In embodiments, the brain activity level measured in the absence of a stimulus is referred to as a baseline brain activity level. Alternatively, brain activity levels may be measured (e.g., quantified) when one or more stimuli are delivered (e.g., an emotional conflict task). In embodiments, the brain activity level measured in the presence of a stimulus is referred to as a brain activity level response. Brain activity levels may be measured simultaneously or sequentially throughout the whole brain, or restricted to specific brain regions (e.g., frontopolar cortex, lateral prefrontal cortex, dorsal anterior cingulate, and anterior insula). In embodiments, the brain activity level is determined relative to a baseline brain activity level taken during a baseline period. The baseline period is typically a period during which a stimulus is not presented or has not been presented for a sufficient amount of time (e.g., great than at least 0.05, 0.1, 0.15, 0.25, 0.5, 1, 2, 3, 4, 5, 10, 15, 30, 60 seconds or more).

A brain activity level may also encompass evaluating functional brain region connectivity. For example, neural activity recorded in a plurality of brain regions may have a specific time course across brain regions that can be correlated to reveal a functional brain connectivity pattern (e.g., at a first time point a first brain regions shows an increase in neural activity and at a second time point a second brain region shows an increase in activity). Thus, in embodiments, a brain activity level is a measurement (e.g., quantification) of a time course of neural activity across a plurality of brain regions. In embodiments, a brain activity level is a sequence of brain region activity levels measured (e.g., quantified) across different brain regions over time. In embodiments, a brain activity level is a functional brain region connectivity pattern.

The term “electroencephalography (EEG)” refers to a non-invasive neurophysiological technique that uses an electronic monitoring device to measure and record electrical activity in the brain.

The terms “treat,” “treatment,” and “treating” in the context of the administration of a therapy to a patient refers to the reduction or inhibition of the progression and/or duration of a disease or condition, the reduction or amelioration of the severity of a disease or condition, and/or the amelioration of one or more symptoms thereof resulting from the administration of one or more therapies.

The term “administering” includes, but is not limited to, oral administration, administration as a suppository, topical contact, intravenous, transdermal, parenteral, intraperitoneal, intramuscular, intralesional, intrathecal, intranasal, rectal, percutaneous, or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. In embodiments, the administering does not include administration of any active agent other than the recited active agent. One preferred route of administration is the oral route.

An “effective amount” is an amount sufficient for a compound to accomplish a stated purpose relative to the absence of the compound (e.g. achieve the effect for which it is administered, treat a disease, reduce enzyme activity, increase enzyme activity, reduce a signaling pathway, or reduce one or more symptoms of a disease or condition). An example of an “effective amount” is an amount sufficient to contribute to the treatment, prevention, delay, inhibition, suppression, or reduction of a symptom or symptoms of a disease or disorder, which could also be referred to as a “therapeutically effective amount.” A “reduction” of a symptom or symptoms (and grammatical equivalents of this phrase) means decreasing of the severity or frequency of the symptom(s), or elimination of the symptom(s). An “effective amount” of a drug can be an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins). Dosages may be varied depending upon the requirements of the patient and the compound being employed. The dose administered to a patient, in the context of the present disclosure, should be sufficient to effect a beneficial therapeutic response in the patient over time. The size of the dose may also be determined by the existence, nature, and extent of any adverse side-effects. Determination of the proper dosage for a particular situation is within the skill of the practitioner.

To determine efficacy of treatment in psychiatric disorders (e.g., depression, major depression) questionnaires (e.g., self-reporting or clinician-administered questionnaires) may be used. Non-limiting examples of questionnaires useful for assessing treatment efficacy in psychiatric disorders (e.g., depression, major depression) include the Hamilton Rating Scale for Depression (HDRS); the Hamilton Rating Scale for Depression 17 item (HDRS₁₇ or HDRS-17); the 21 item HDRS (HDRS₂₁); the 24 item HDRS (HDRS₂₄); the Quick Inventory of Depressive Symptoms (QIDS); the Patient Health Questionnaire (PHQ-9); the Cognitive and Physical Functioning Questionnaire (CPFQ); the Mood and Symptom Questionnaire subscale scores for Anxious Arousal, Anhedonic Depression, and General Distress; the Montgomery-Asberg Depression Scale (MADRS); the Beck Depression Inventory; the Clinical Global Impressions (GCI) scale); and the Snaith-Hamilton Pleasure Scale (SHAPS). Questionnaires may be completed prior to, during, and following treatment, and changes in the scores may be used to determine treatment efficacy. In embodiments, the HDRS₁₇ is used to determine treatment efficacy. In embodiments, the HDRS is used to determine treatment efficacy. In embodiments, the HDRS₂₁ is used to determine treatment efficacy. In embodiments, the HDRS₂₄ is used to determine treatment efficacy. In embodiments, the QIDS is used to determine treatment efficacy. In embodiments, the Mood and Symptom Questionnaire subscale scores for Anxious Arousal, Anhedonic Depression, and General Distress are used to determine treatment efficacy. In embodiments, the MADRS is used to determine treatment efficacy. In embodiments, the Beck Depression Inventory is used to determine treatment efficacy. In embodiments, the clinical global impression (CGI) scale is used to determine treatment efficacy. In embodiments, treatment efficacy is determined by measuring (e.g., quantifying) a change in the HDRS₁₇ score. In embodiments, treatment efficacy is determined by measuring (e.g., quantifying) a change in the HDRS₂₁ score. In embodiments, treatment efficacy is determined by measuring (e.g., quantifying) a change in the HDRS score. In embodiments, treatment efficacy is determined by measuring (e.g., quantifying) a change in the HDRS₂₄ score. In embodiments, treatment efficacy is determined by measuring (e.g., quantifying) a change in the QIDS score. In embodiments, treatment efficacy is determined by measuring (e.g., quantifying) a change in the Mood and Symptom Questionnaire subscale scores for Anxious Arousal, Anhedonic Depression, and General Distress. In embodiments, treatment efficacy is determined by measuring (e.g., quantifying) a change in the MADRS score. In embodiments, treatment efficacy is determined by measuring (e.g., quantifying) a change in the Beck Depression Inventory score. In embodiments, treatment efficacy is determined by measuring (e.g., quantifying) a change in the CGI scale. In embodiments, treatment efficacy is determined by measuring (e.g., quantifying) a score on a questionnaire as described herein during a baseline period prior to treatment to a score on a questionnaire as described herein reported 1, 2, 3, 4, 6, 8 or more weeks after commencing treatment or terminating treatment.

Treatment may result in a reduction of symptoms (e.g., a response) or in remission. In embodiments, a reduction in symptoms is referred to as a response. In embodiments, a response is a 50% or greater decrease in symptoms. A response (e.g., a 50% or greater decrease in symptoms) to treatment may be determined by measuring (e.g., quantifying) a change in a score as described herein, including embodiments thereof, on a questionnaire as described herein, including embodiments thereof. In embodiments, remission is a score of 7 or less at endpoint on the HDRS₁₇. In embodiments, remission is a score of 7 or less at endpoint on the HDRS. In embodiments, remission is a score of 10 or less on the HDRS₂₄. In embodiments, remission is a score of 5 or less on the QIDS. In embodiments, remission is a score of 9 or less on the MADRS.

The term “poor cognition” (or “cognitively poor”), unless otherwise defined, refers to a subject having cognitive function, as measured by one or more tests of cognitive function, below that of the 50^{th} percentile of healthy subjects of similar age (z-score<0). Z scores reflect a transformation of cognitive task performance relative to a healthy subject distribution, which may account for factors such as age, education and gender in that transformation. A z score below zero indicates performance for that subject that is below the 50^{th} percentile of similar healthy subjects, while a z score above zero indicates performance that is above the 50^{th} percentile of similar healthy subjects. For example, the subject may have a cognitive score below the 50^{th} percentile of a similar healthy subject with a z-score less than zero, less than z=−0.5, z=−1, or z=−2 (e.g., with a z-score of from about −0.5 to about −1 or about −2, or a z-score of from about −1 to about −2).

Cognition can be assessed by methods known in the art, including those described in DSM-5 (see, e.g., pages 593-595). For instance, cognition can be measured by a simple reaction time test, choice reaction time test, one back working memory task, visual learning task, or any combination of any of the foregoing. In one embodiment, the cognitive ability of a subject is measured with a Cogstate Brief Batter as described in Maruff et al., Arch Clin Neuropsychol. 2009, 24 (2): 165-78, which is hereby incorporated by reference. Tests of cognitions (such as to assess information processing speed, working memory, learning, and attention) include, but are not limited to, Digit symbol substitution task, Oddball task, Flanker task, Wisconsin card sort task, Trail making task, Corsi Block task, Digit Span task, Reverse Digit Span task, Verbal Learning and Memory task, and Verbal Fluency task. Reduced information processing speed, slow decision making or difficulty making decisions may be diagnosed by tests that assess reaction times or performance under speed-based task instructions (e.g., reduced number of correct symbols in a digit symbol substitution task or reduced verbal fluency in a fixed amount of allotted time), such as those described in J. DeLuca and J. H. Kalmar, Information Processing Speed in Clinical Populations, Taylor & Francis Group (2008), which is hereby incorporated by reference. Decision making (including slow decision making and difficulty making decisions) can be assessed by the performance of tasks that assess process of deciding in the face of competing alternatives (e.g., simulated gambling) (DSM-5, p. 593).

Reductions in attention can be assessed by:
(1) for sustained attention: maintenance of attention over time (e.g., pressing a button every time a tone is heard, and over a period of time),
(2) for selective attention: maintenance of attention despite competing stimuli and/or distractors: hearing numbers and letters read and asked to count only letters, and
(3) for divided attention: attending to two tasks within the same time period: rapidly tapping while learning a story being read. Processing speed can be quantified on any task by timing it (e.g., time to put together a design of blocks; time to match symbols with numbers; speed in responding, such as counting speed or serial 3 speed).

Reductions in working memory can be assessed by the ability to hold information for a brief period and to manipulate it (e.g., adding up a list of numbers, repeating a series of numbers or words backward or repeating a sequence of actions).

Reductions in memory can also be assessed by the following methods (in addition to the working memory assessment method described above):
(1) Immediate memory span: Ability to repeat a list of words or digits.
(2) Recent memory: Assesses the process of encoding new information (e.g., word lists, a short story, or diagrams). The aspects of Assesses the process of encoding new information (e.g., word lists, a short story, or diagrams). The aspects of recent memory that can be tested include 1) free recall (the person is asked to recall as many words, diagrams, or elements of a story as possible); 2) cued recall (examiner aids recall by providing semantic cues such as “List all the food items on the list” or “Name all of the children from the story”); 3) recognition memory (examiner asks about specific items—e.g., “Was ‘apple’ on the list?” or “Did you see this diagram or figure?”); and 4) recall of an original list of items or words after presentation of a distractor list of items or words. Other aspects of memory that can be assessed include semantic memory (memory for facts), autobiographical memory (memory for personal events or people), and implicit (procedural) learning (unconscious learning of skills).

The term “slow cognition”, unless otherwise defined, refers to a subject having slow cognitive function (longer time to respond), as measured by one or more tests of information processing speed (such as a simple reaction time test or choice reaction time test), below the 50^{th} percentile of a healthy subject of similar age (z-score<0).

Processing speed can be quantified on any task by timing it (e.g., time to put together a design of blocks; time to match symbols with numbers; speed in responding, such as counting speed or serial 3 speed).

Reduced learning can be assessed by the methods described above for immediate memory span and recent memory.

In one embodiment, the cognitive impairment, poor or slow cognition or difficulty making decisions is due, at least in part, to reduced attention, memory, learning, working memory, or any combination of any of the foregoing.

As used herein, the terms “subject” and “patient” are used interchangeably and refer to a human patient unless indicated otherwise.

Suitable antidepressants for concurrent therapy includes, but is not limited to, (i) monoamine oxidase inhibitors (MAOIs), (ii) tricyclic antidepressants (TCAs) (such as amitriptyline, imipramine, clomipramine, and desipramine), (iii) serotonin and norepinephrine reuptake inhibitors (SNRIs) (such as venlafaxine, duloxetine, milnacipran, sibutramine, SEP-227162, or LY 2216684), (iv) selective serotonin reuptake inhibitors (SSRIs) (such as escitalopram, fluoxetine, fluvoxamine, sertraline, citalopram, vilazodone, and paroxetine), (v) atypical antidepressants (such as agomelatine, mianserin, mirtazapine, nefazodone, opipramol, tianeptine, and trazodone), and (vi) norepinephrine and dopamine reuptake inhibitors (NDRIs) (such as bupropion, amineptine, prolintane, dexmethylphenidate, and pipradrol).

Suitable mood stabilizers include, but are not limited to, lithium carbonate, divalproex sodium, valproic acid, valproate semisodium, sodium valproate, tiagabine, levetiracetam, lamotrigine, gabapentin, carbamazepine, oxcarbazepine, topiramate, zonisamide, aripiprazole, risperidone, olanzapine, quetiapine, asenapine, paliperidone, ziprasidone, lurasidone, verapamil, clonidine, propranolol, mexiletine, guanfacine and omega-3 fatty acids.

### Example 1—Phase 2 Clinical Trial with NSI-189

A retrospective analysis of the 220-patient phase 2 study of NSI-189 discussed above was performed. In the study, patients having major depressive disorder were randomized to receive a total daily dose of 40 mg or 80 mg of NSI-189 or placebo in stage 1 (first 6 weeks) of the clinical trial design. Patients receiving placebo and who failed to respond to placebo were then re-randomized to 40 mg, 80 mg or placebo in stage 2 (second 6 weeks) of the trial. The remaining of patients continued on their treatment for stage 2 of the trial. Treatment in either stage was given for six weeks. Patients were eligible for study participation if they were between the ages of 18-60 years, with current major depressive disorder of at least 8 weeks duration according to the DSM-5, as diagnosed by the Structured Clinical Interview for the DSM-5 clinical trial version (SCID-5-CT) during the screen and remote assessment visits, and if they were scored at least 20 at screen, remote assessment, and baseline visits on the Montgomery-Asberg Depression Rating Scale (MADRS). The phase 2 study collected cognitive task performance data prior to treatment, and then again after both stage 1 and stage 2 of the treatment protocol. The purpose of doing so was to determine whether treatment with this compound results in improvement in cognitive functioning, as measured by a variety of behavioral measures.

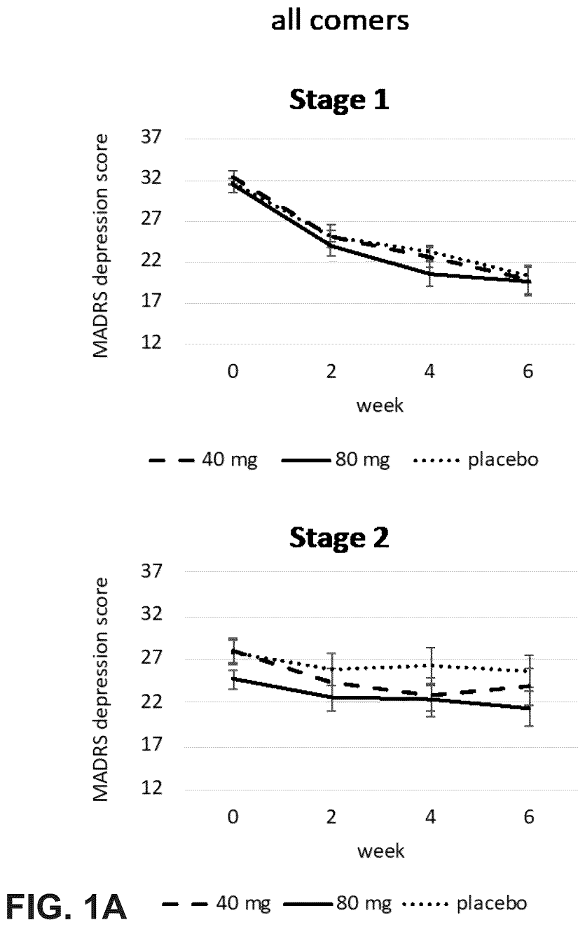

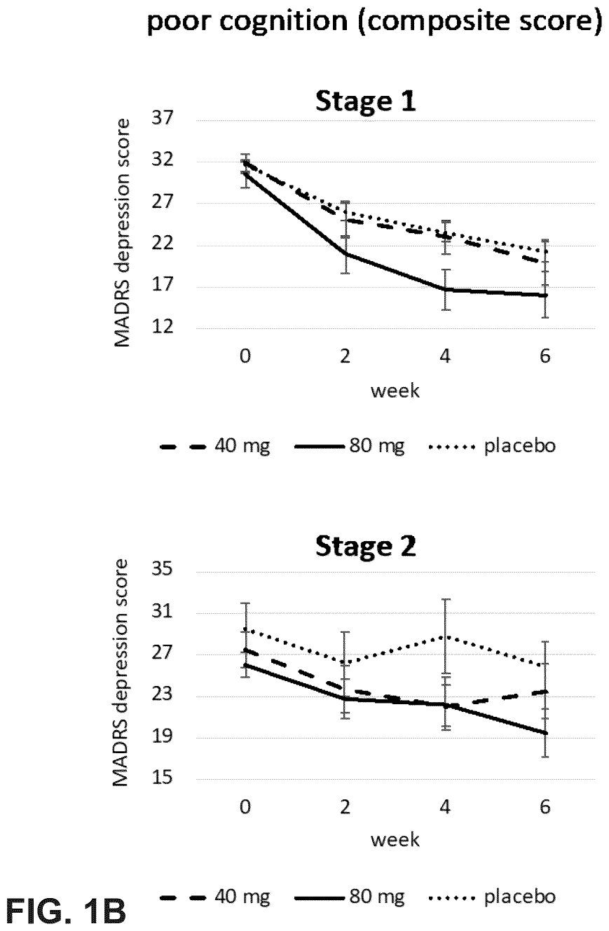

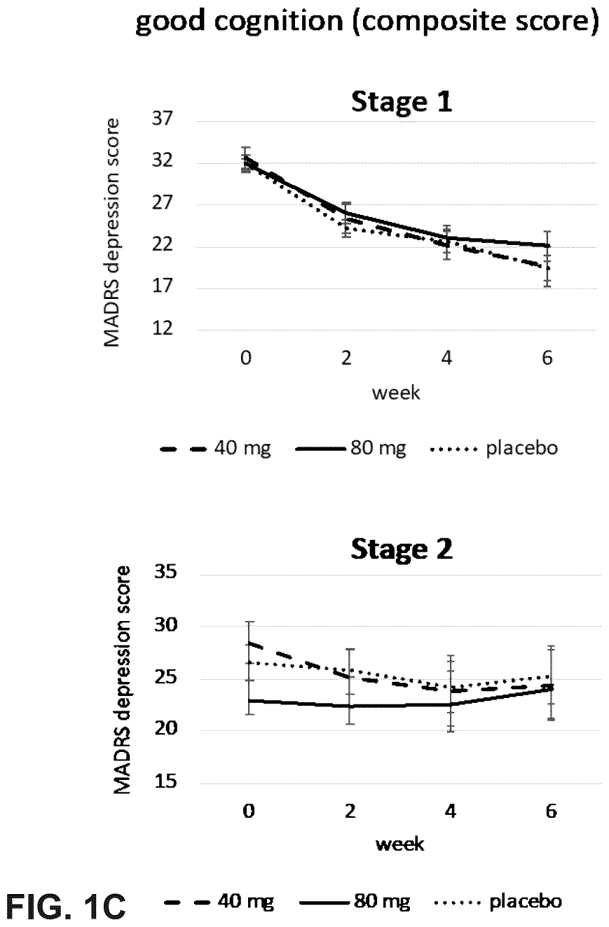

Prior to the inventor's retrospective analysis, there was no consideration as to using behavioral measures to predict treatment outcome nor was there any analysis conducted to this effect. There was no consideration of using cognitive performance as a predictor but only as an outcome measure. The analyses described herein focus on the four cognitive task measures included in the Cogstate battery, as these data are available as z-scores wherein each individual's performance was normalized to that in a large healthy population. The tasks in this battery included a simple reaction time (RT) task, a choice RT task, a one-back working memory task, and a visual learning task. Moreover, given the conversion to z-score, a composite cognitive task performance score could be computed by averaging the z-scores for each of the four tasks in the battery. Analyses focused on the MADRS primary outcome of the study, with supporting evidence subsequently produced by examining the HDRS key secondary outcome, as well as the CGI. Statistical analyses focused first on the complete trial (incorporating both stage 1 and stage 2 outcomes), using the SPCD analytic approach reported in the original study and thus aligned could be compared with the original statistical analysis plan. We used linear mixed models to predict change from baseline (at 2, 4, and 6 weeks) in clinical scores and included covariates for time, time x group, time x baseline MADRS and baseline MADRS. These covariates likewise were used in the original study, and thus results here can be compared with the all-comer results reported previously. Statistical significance was thus assessed on the primary outcome (MADRS change from baseline at 6 weeks across the SPCD design) and similar secondary outcomes (HDRS and CGI change from baseline at 6 weeks). Follow-up analyses focused on specific aspects outside of the SPCD analytic framework to answer additional questions. The change in MADRS scores in the all corner (i.e. original sample) analysis is shown in FIG. 1A for comparison to subgroups as defined by cognitive task performance.

To examine the effect of baseline cognition on clinical outcomes, we split individuals at the mean of the z-score for each task separately, as well as at the mean of the average z-score across all tasks (thus a cognition composite score). This generates two groupings for each test, which we termed poor cognition (below mean) or good cognition (above mean). The poor cognition group can also be termed to be cognitively impaired. We did the first analyses with the cognitive composite score, followed by similar analyses for each of the cognitive tests performed. The composite score mean at which the cut-off between good and poor cognition groups was performed was at z=−0.32. Notably, this mean is in line with expectations of moderately-impaired cognitive task performance in depression in general (12, 13).

Surprisingly, it was found that while the original all-comer analysis failed to find statistical significance on the primary MADRS outcome, we found a robust and statistically significant effect for the 80 mg arm versus placebo on change in MADRS scores in the poor cognition group using the cognitive composite score (SPCD analysis p-0.014; FIG. 1B). Though the 40 mg group was visually intermediate in outcome to the 80 mg and placebo group, this effect was not significant (p=0.39; FIG. 1B). By contrast, no significant differences were found between either drug arm and placebo in the good cognition group (p=0.25, FIG. 1C). Thus, not only does cognitive task performance successfully differentially predict outcome between drug and placebo, this appears to be substantially stronger for the 80 mg treatment relative to 40 mg, which runs counter to expectations from prior treatment main effect analyses of the data as discussed above (11). This is also surprising since poor cognition was previously reported to be consistently predictive of poor response to SSRIs and SNRIs in multiple studies of depressed patients. Groves et al., Front. Psychiatry 9:382, 2018; Etkin et al., Neuropsychopharmacology 40 (6): 1332-1342, 2015.

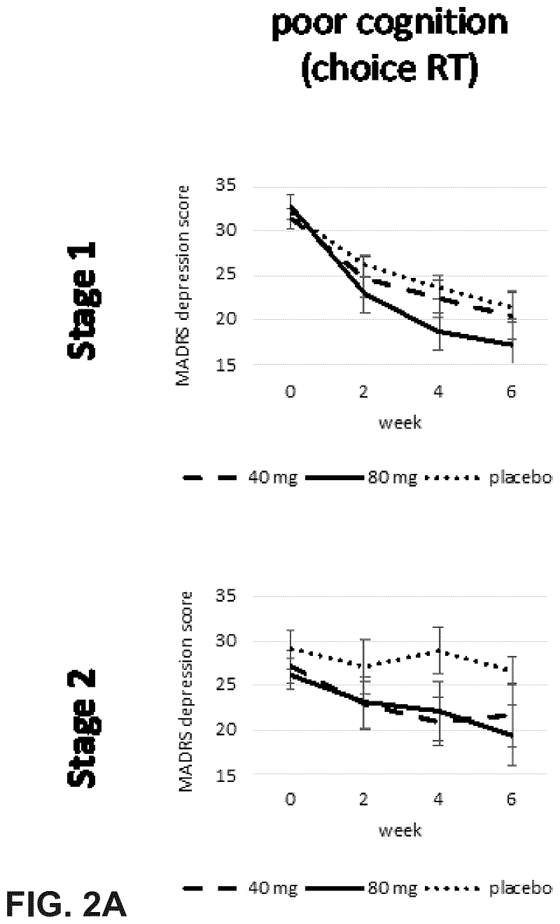

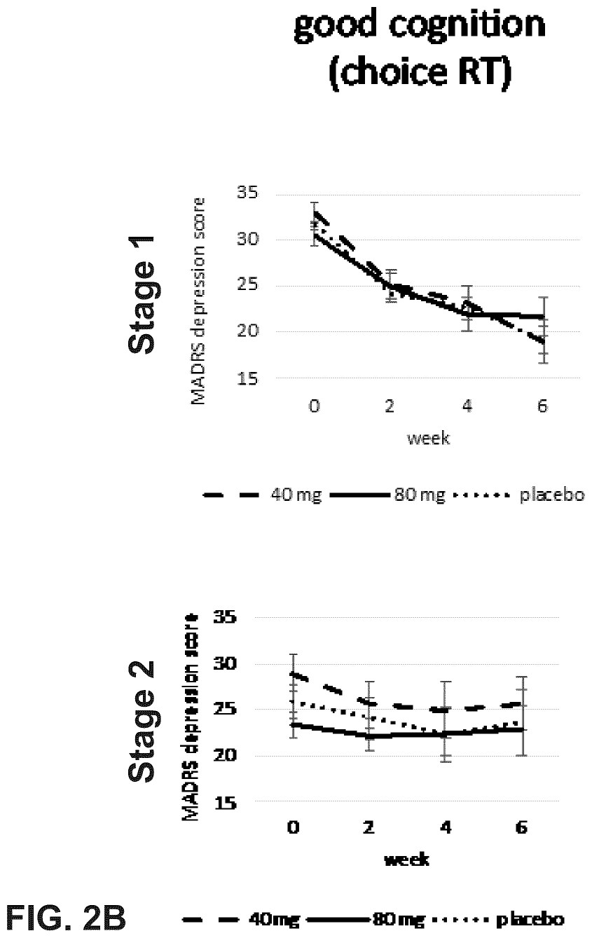

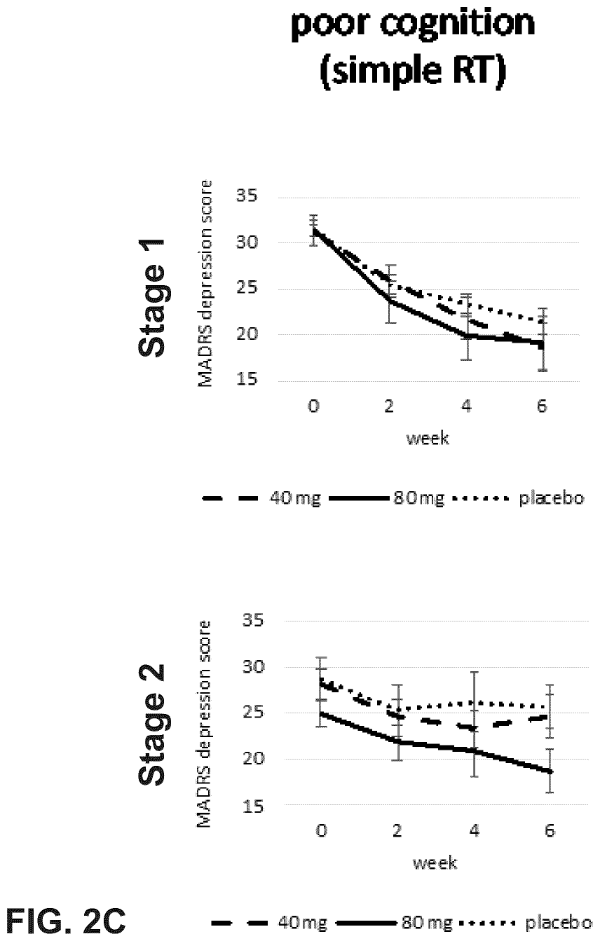

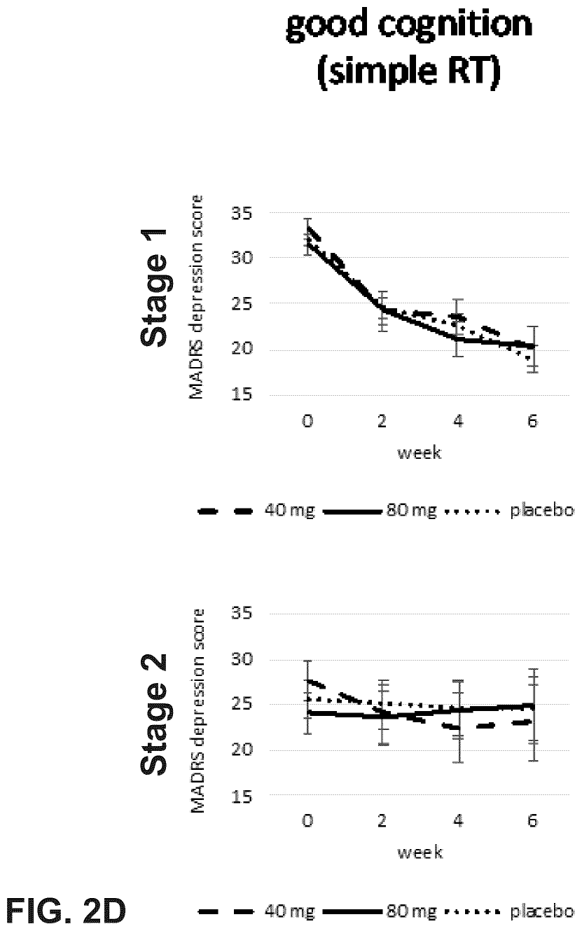

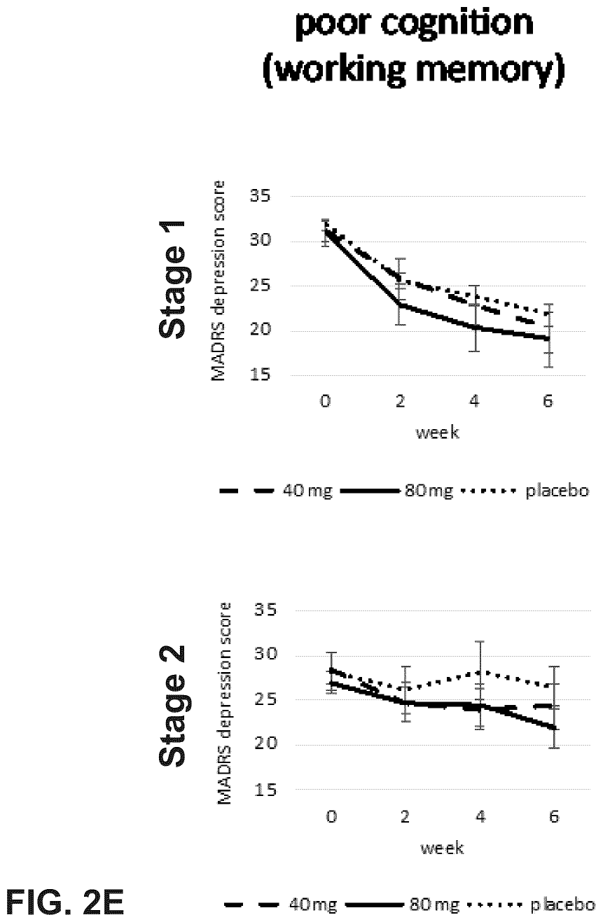

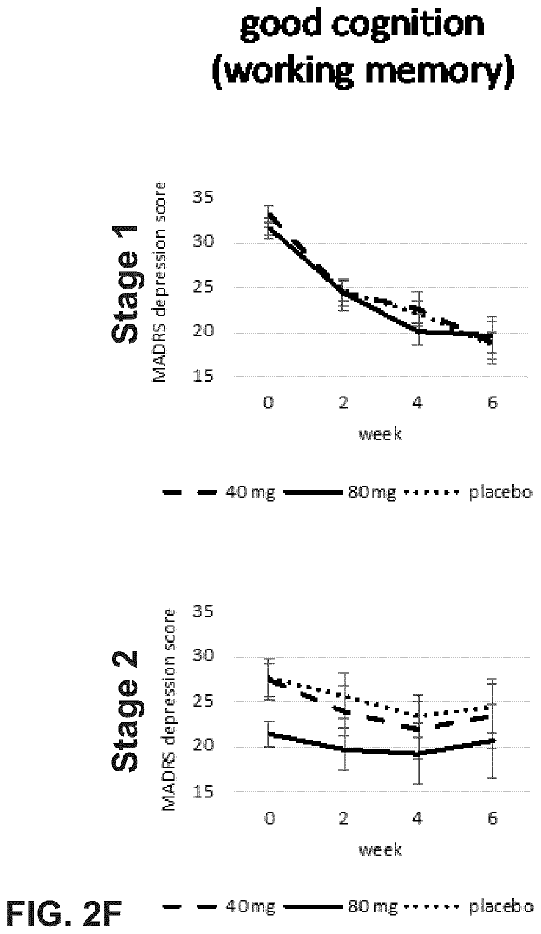

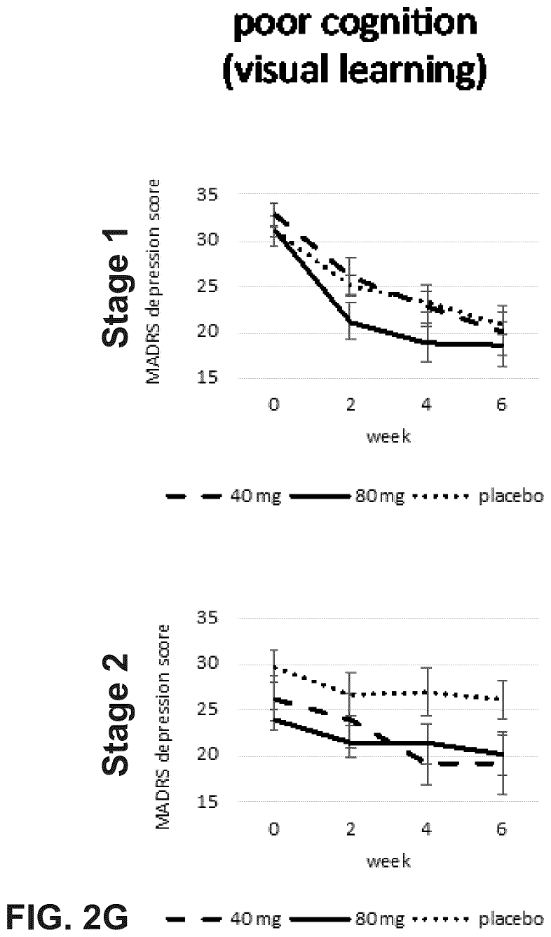

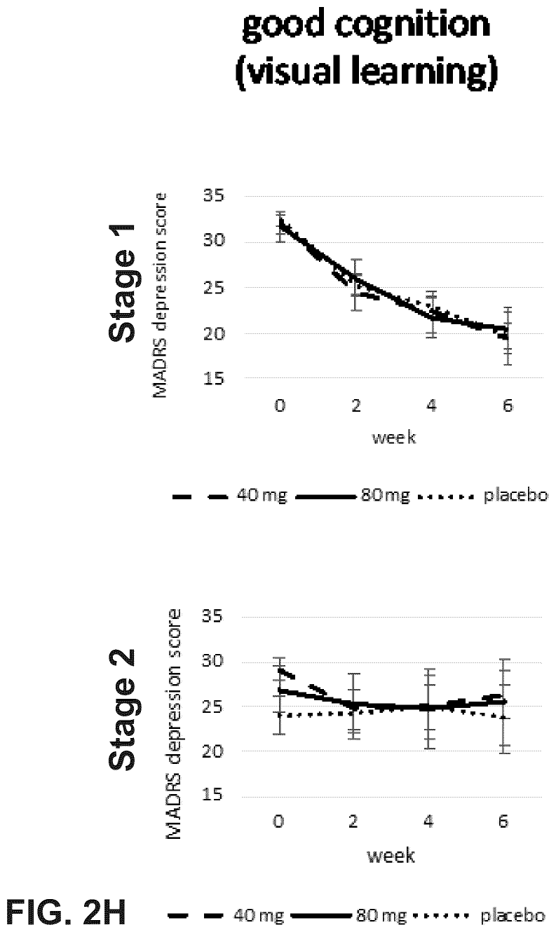

We next examined the change in MADRS scores when dividing patients into good and poor cognition groups using groupings determined from each of the cognitive tests separately (namely, choice RT, simple RT, working memory and visual learning). These analyses are shown in FIGS. 2A-2H. A similar pattern of change was observed for the poor cognition group using any of the cognitive tests, such that improvement in the 80 mg group was visually superior to the placebo group, with 40 mg typically intermediate. Applying the SPCD statistical analyses, we found that the 80 mg group resulted in a robust and significant improvement over placebo for poor cognition as defined using the choice RT task (p=0.005), with trend-level significance for the simple RT task grouping (p=0.09) and one-back working memory (p=0.08).

It was additionally tested whether the effects above for the 80 mg versus placebo comparison depend on the cutoff used to define the poor cognition group on the choice RT task. 80 mg was found to have significant superiority over placebo when using a more liberal definition of cognitive impairment at z=0 (p=0.007) and at a more stringent definition at z=−0.5 (p=0.009). Using a far more lenient definition at z=+0.5 (which included some good cognition patient), however did not yield a significant effect (p=0.12). Thus, the significant antidepressant effects of NSI-189 map broadly onto the concept of poor or slow cognition, difficulty making decisions or cognitive impairment in depression, and do not depend on selection of a precise threshold for defining this construct.

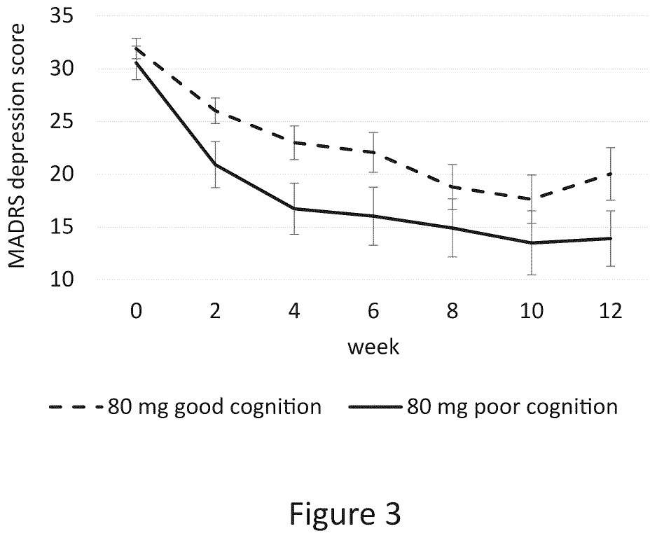

Because patients who received drug in stage 1 continued on drug during stage 2 of the trial, we could also examine the durability of the difference in treatment outcome as a function of cognition. FIG. 3 shows that the benefit in the 80 mg arm in the poor cognition compared to good cognition patients (using the composite score) was stable and consistent throughout all 12 weeks of treatment for these patients.

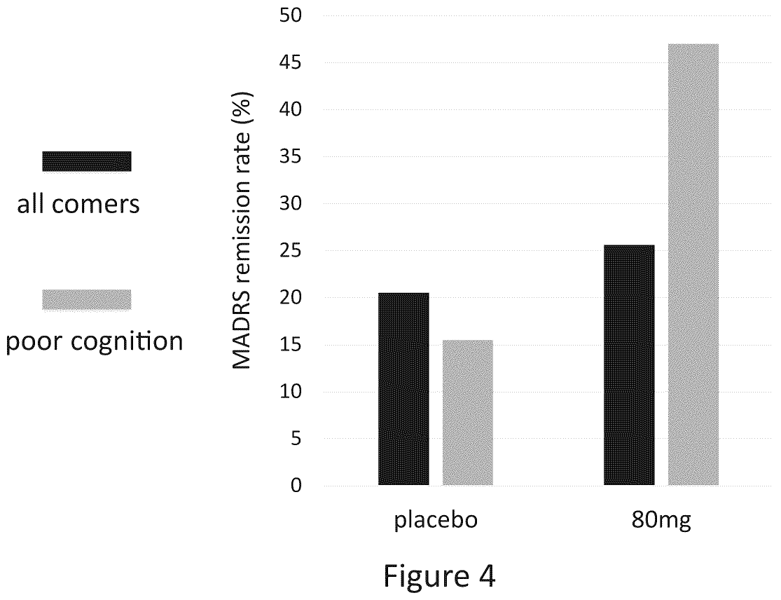

We next assessed clinical significance of the above findings by quantifying remission (a score of 10 or less on the MADRS) after 6 weeks of treatment in stage 1. 80 mg results in a 47% remission rate in the poor cognition patients (using the composite score) relative to an only 16% remission rate for placebo (chi-square p=0.017; FIG. 4). The magnitude of this striking difference in clinical outcome can be quantified as an odds ratio for drug response, which increases from 1.3 in the all-comer sample to 4.7 in the poor cognition subpopulation. Likewise, the number needed to treat to achieve one more remission drops from 19.6 in the all-comer sample to 3.2 in the poor cognition subpopulation. These results are particularly compelling in the context of extensive prior work showing that patients who are more cognitively impaired on measures such as those used here are less responsive to existing antidepressants (14). This patient population is therefore particularly under-served by existing antidepressant agents. Thus, our surprising findings support the unique potential of NSI-189 in the treatment of patients with depressive symptoms and cognitive impairment. No benefit in terms of remission rate was observed for the good cognition group. In the 80 mg arm, the remission rate in poor cognition patients was also significantly better than that of good cognition patients (47% vs. 12%; chi-square p-0.014).

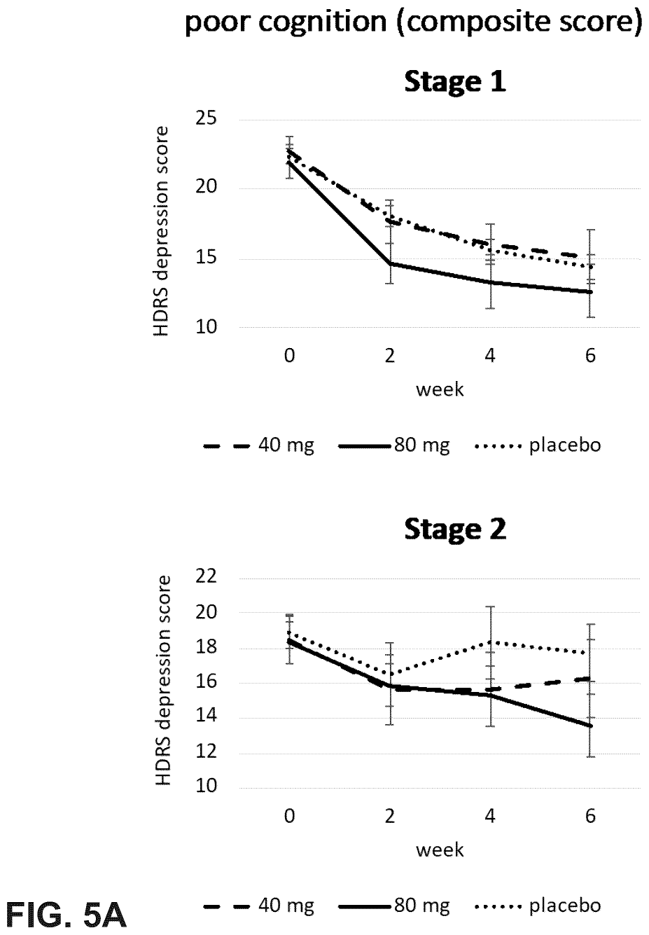

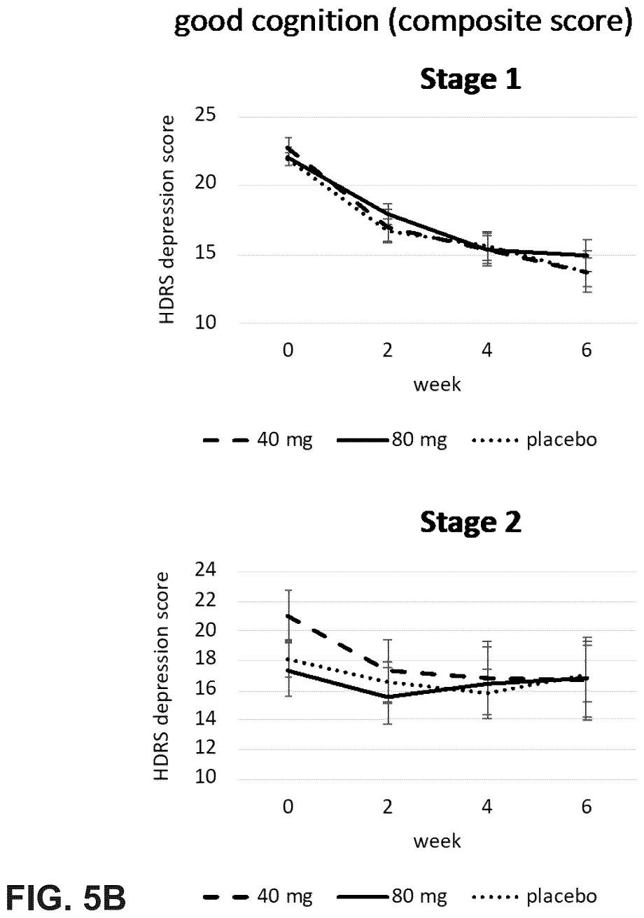

As another way to determine the clinical significance of these findings, shown in FIG. 5 is symptom change on the HDRS in poor and good cognition patients based on the composite cognitive task performance score. In line with the results shown for the MADRS, results for the HDRS likewise demonstrate significantly greater efficacy of 80 mg over placebo in poor cognition patients (p=0.038). Similarly, when defining poor cognition based on the choice RT task, a robust and significant effect of the 80 mg arm was observed relative to placebo (p=0.008). Hence, both of the gold-standard depression scales accepted by the FDA demonstrate the same pattern of change.

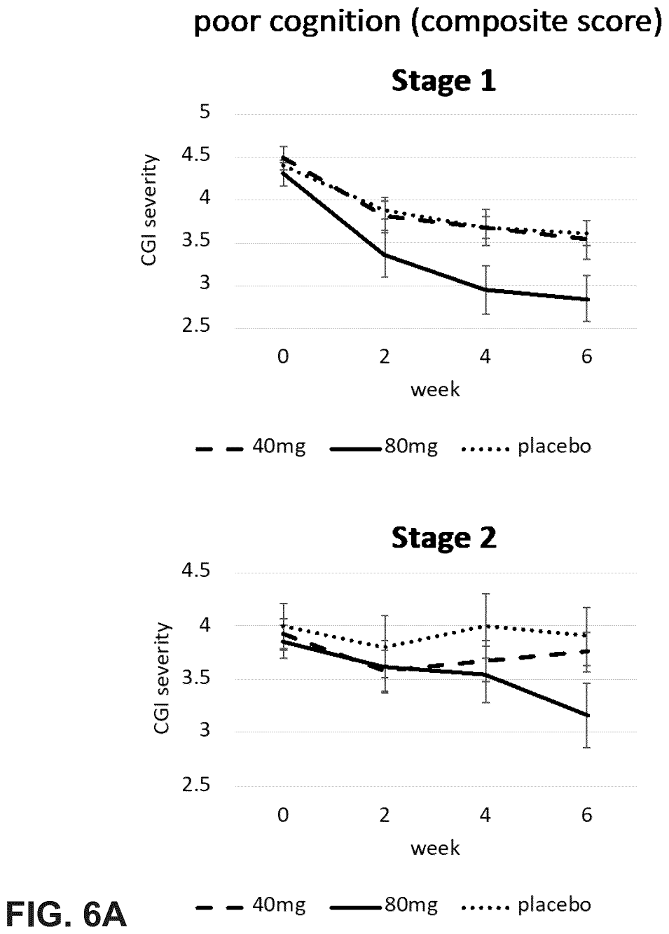

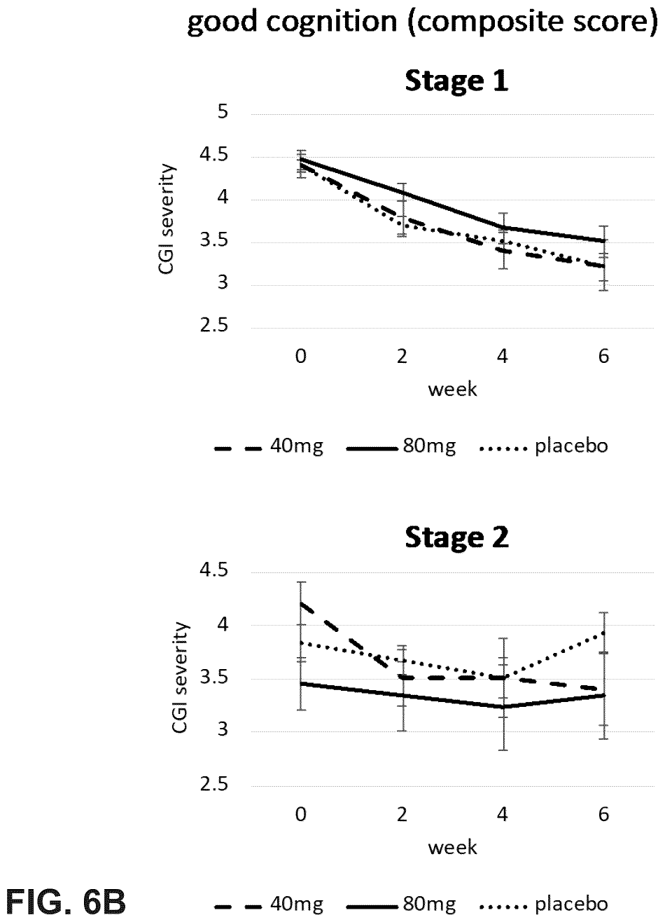

Likewise, the same pattern was seen on the Clinical Global Impression (CGI) severity scale, which is the principle secondary outcome measure accepted by FDA (FIG. 6). Specifically, 80 mg was significantly more effective than placebo in poor cognition patients (p=0.002), but not in good cognition patients. Doing the same analysis using choice RT to define poor cognition patients similarly revealed a robust and significant advantage of 80 mg over placebo (p=0.0002).

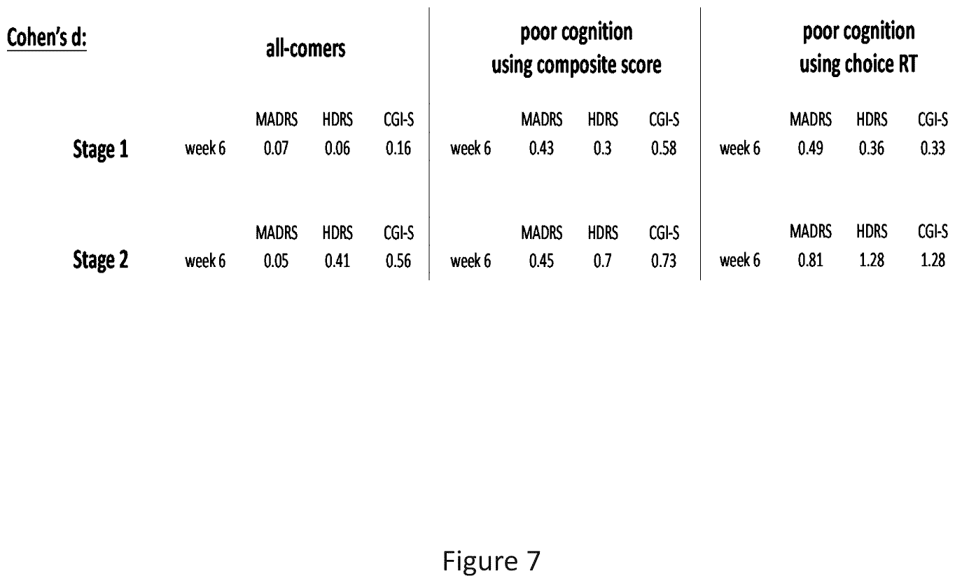

To quantify the magnitude of clinical improvement in poor cognition, as well as its clinical significance, shown in FIG. 7 are Stage 1 and Stage 2 Cohen's d effect sizes for poor cognition groups as defined by either the cognitive composite score or choice RT for each of the outcomes above (MADRS, HDRS, CGI), along with the all-comer analysis. Effect sizes are given for the 80 mg versus placebo comparison at week 6. As is evident from this figure, effect sizes increase substantially in the poor cognition subpopulation of patients relative to the all-comer sample, at times reaching extremely large effect sizes. Typical interpretations of Cohen's d effect sizes are that 0.3 is small, 0.5 is medium, and 0.7 is large.

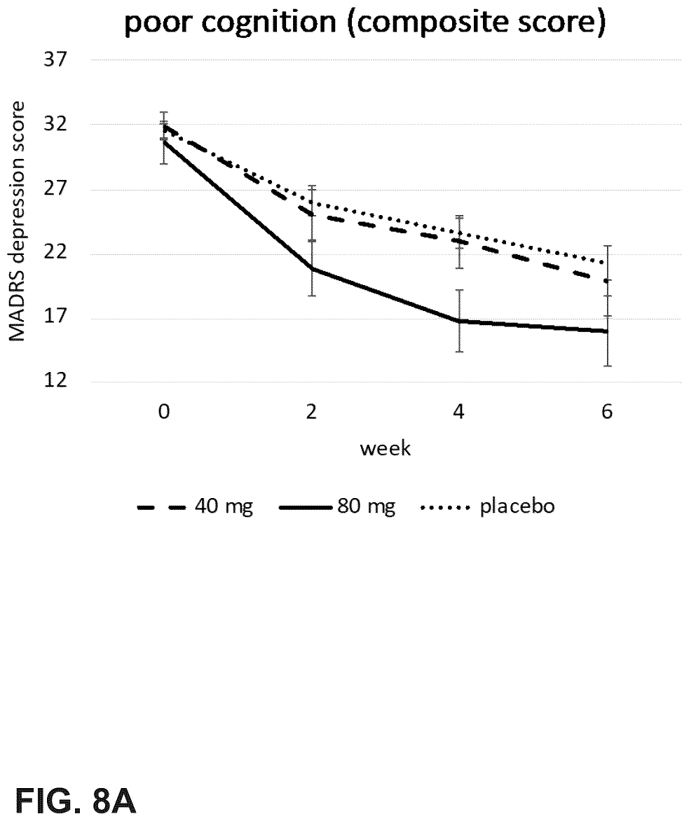

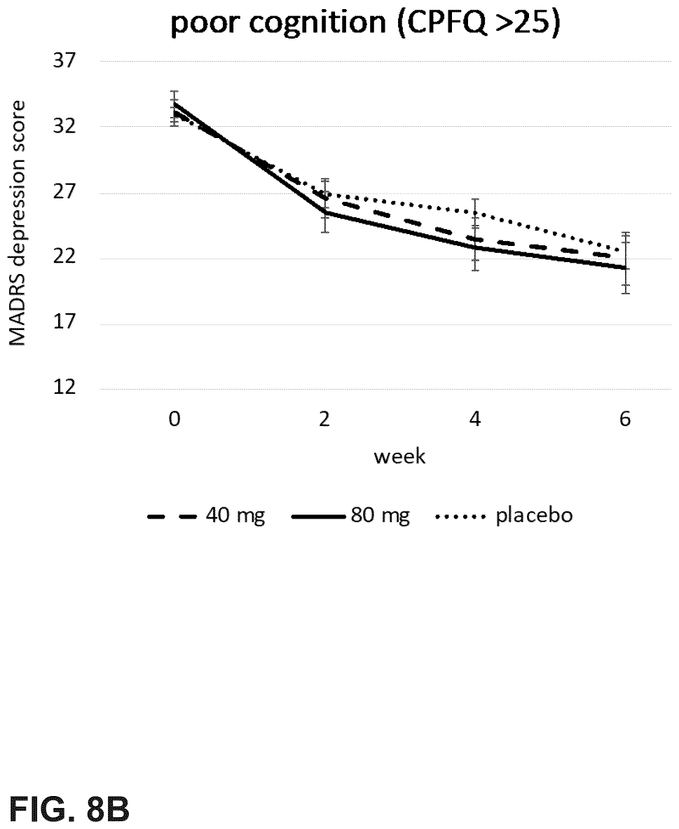

Whether poor cognition could be defined in an alternative manner was tested, namely by asking patients to rate their cognition on a self-report questionnaire. One such questionnaire is the Cognitive and Physical Functioning Questionnaire (CPFQ) (15) This scale is notable as it was featured in analyses for the antidepressant vortioxetine in its approval by both the FDA and the European Medicines Agency (EMA). In those analyses, poor subjective cognition was defined as a CPFQ score of >25, and thus whether cognitive impairment based on the CPFQ could identify individuals who preferentially benefit from treatment with NSI-189 was examined. As shown in FIG. 8, which contrasts poor cognition defined using the composite cognitive task performance score (as shown earlier) with that defined by the CPFQ, no difference is seen between treatment arms in patients with poor self-reported cognition. Thus, surprisingly, not only is poor objective cognition a predictor of better drug response, but this impairment must be measured through performance of cognitive tasks such as those used here (simple RT, choice RT, working memory or visual learning). The objective measurement cannot be replaced by use of patient-reported cognitive symptoms. This furthermore did not depend on the cutoff used on the CPFQ to define poor cognition patients as multiple other cutoffs were used to define poor subjective cognition on the CPFQ and none were predictive of drug response.

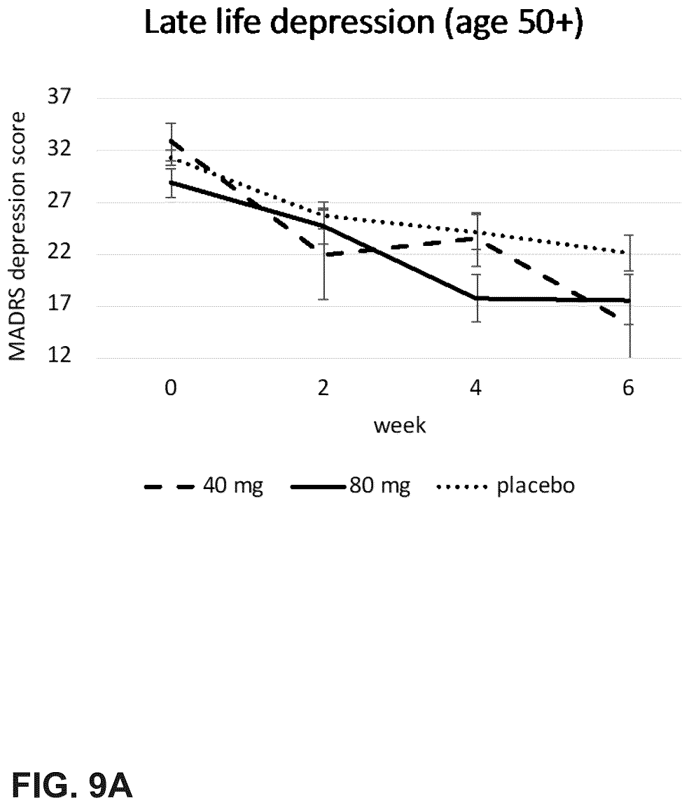

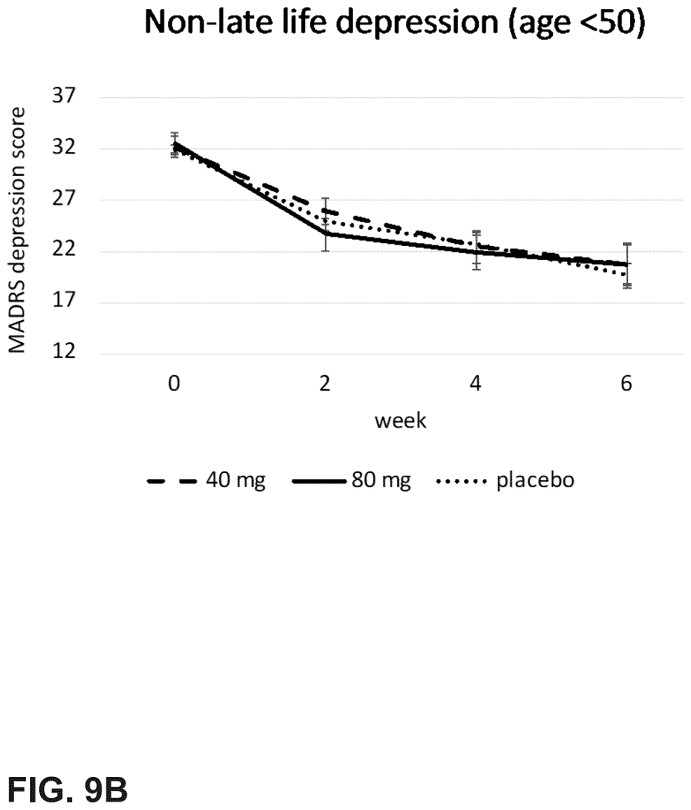

Building on the finding of poor cognition predicting better treatment outcome, late-life depression (LLD) was next looked at. LLD was defined as patients who were 50 years or older, compared to non-LLD patients who were younger than 50. LLD is associated with cognitive impairment (16), and hence LLD patients were hypothesized to experience greater symptom improvement for 80 mg over placebo relative to non-LLD patients. As seen in FIG. 9, MADRS depression score improvement was greater with 80 mg over placebo in the LLD group, but indistinguishable between the two arms in the non-LLD group.

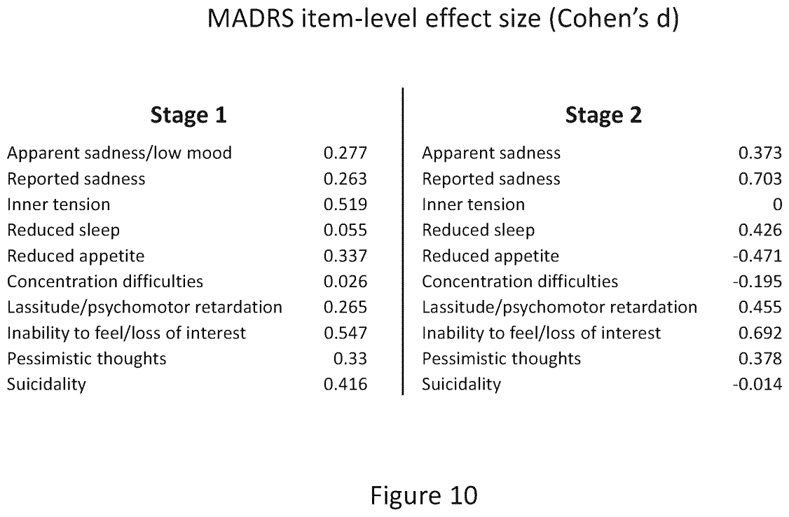

To better understand the clinical effects of the drug at 80 mg in cognitively impaired patients, the Cohen's d effect size at each stage for each item of the MADRS at six weeks of treatment for the comparison of the 80 mg and placebo groups were quantified (see FIG. 10). As can be seen, effect sizes are not equal across all items. Rather, across both stages, there are notable effects on items that reflect sadness (item 1), inability to feel and loss of interest in activities (also referred to as anhedonia; item 8), psychomotor retardation and lassitude (also referred to as loss of motivation; item 7), and pessimistic thoughts (item 9). As such, these results suggest that individuals whose depressive symptoms are more heavily predominated by the types of items on which a greater drug effect is seen are more likely to respond to the drug. For example, individuals with particularly high anhedonia or psychomotor retardation are particularly responsive to the drug. In a similar manner, other disorders in which depressive symptoms are prominent and poor cognition is likewise prevalent may be particularly sensitive to the drug. This includes disorders such as post-traumatic stress disorder (PTSD), bipolar depression, substance use disorder and schizophrenia. For example, according to the DSM-5, PTSD includes symptoms such as strong negative beliefs about self/other, guilt/shame, loss of interest, feeling distant, difficulty experiencing positive feelings and difficulty concentrating. These map well onto items that show particularly high sensitivity to the drug in cognitively impaired individuals. PTSD is also associated with frequent cognitive impairments similar to those examined here (17). Bipolar depression is assessed with the same symptom measures as those used for major depression (e.g., MADRS and HDRS) and thus cognitively impaired patients may be particularly sensitive to the drug in a manner similar to major depression patients with poor cognition. Substance use disorders, while primarily dominated by the consequences of maladaptive substance use and dependence, often includes depressive symptoms. These symptoms may, in fact, drive the patient to engage in further substance abuse as an attempt to self-medicate their addiction. Various substance use disorders are also associated with poor cognition (18). Thus, patients with substance use disorders and poor cognition may be particularly sensitive to the effects of the drug. With respect to schizophrenia, DSM-5 recognizes negative symptoms (e.g. anhedonia, decreased emotional expression or experience) as a prominent part of the syndrome of schizophrenia beyond positive symptoms (e.g. hallucinations and delusions) and disorganization of thought or behavior. Patients with schizophrenia also have negative cognitive biases (e.g., pessimistic thoughts). Schizophrenia is also a disorder of profound and frequent cognitive impairment. Thus, the negative symptoms of schizophrenia, and in particular in patients with poorer cognition, may identify patients with schizophrenia who are particularly sensitive to the effect of the drug.

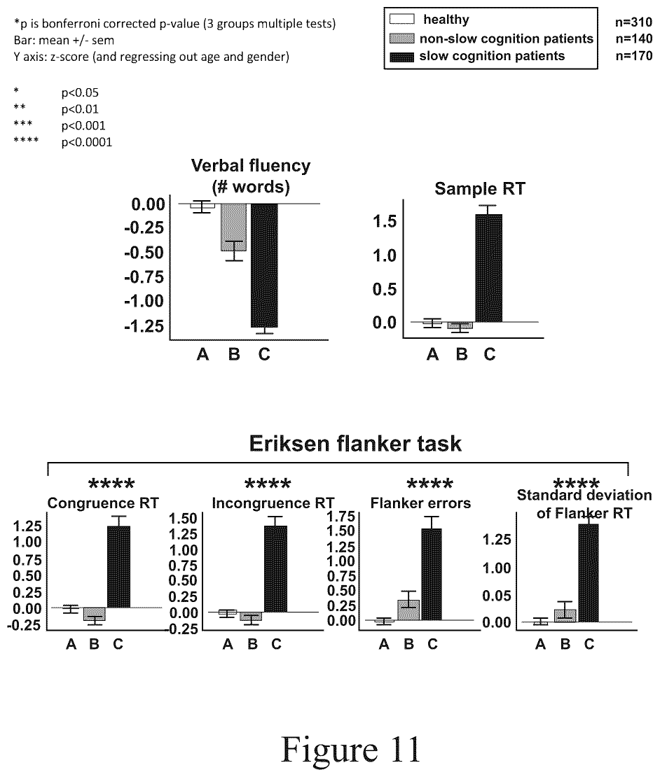

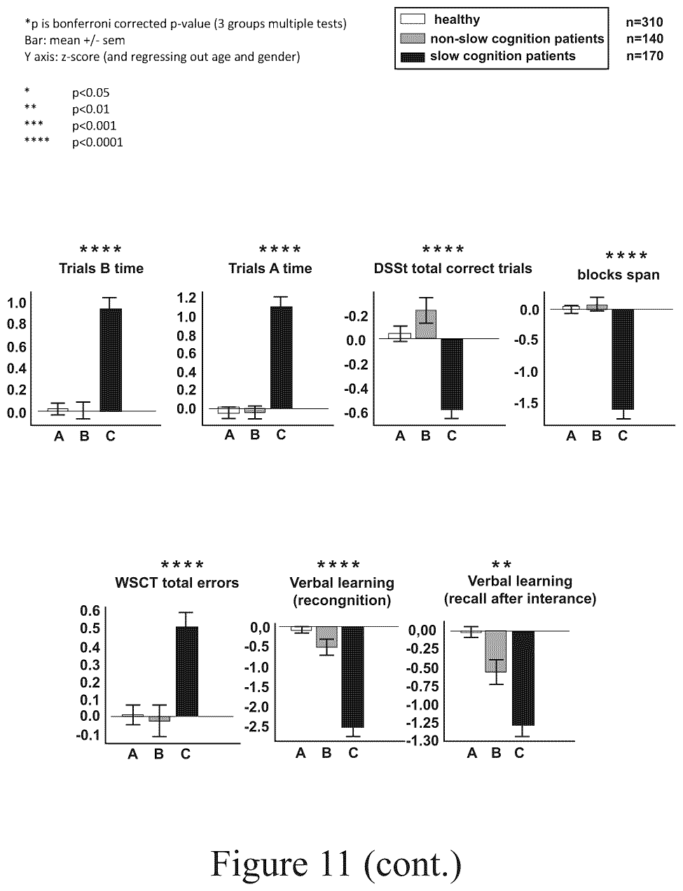

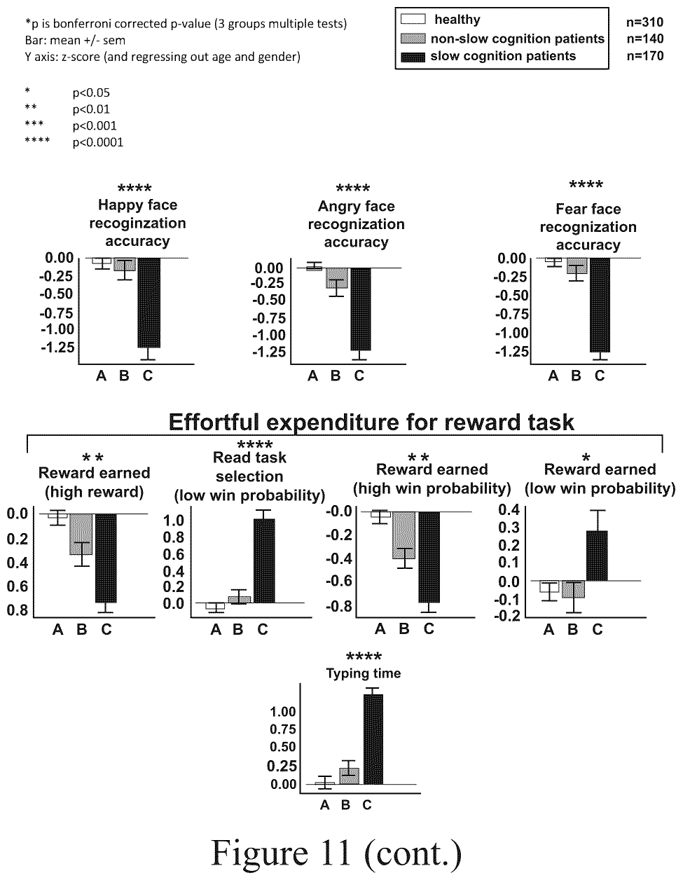

To better understand, the inventors collected data on a broader cognitive battery in a group of 310 psychiatrically healthy individuals and 310 individuals with moderate to severe depression. This battery included the following tasks: simple reaction time task, choice reaction time task, Eriksen flanker task, digit symbol substitution task, Corsi block task, verbal learning and memory task, a typing-adapted version of a verbal fluency, trail making test, Wisconsin card sorting task, delay discounting task, effortful expenditure for reward task and the facial emotion recognition test. All behavioral data were converted to z-scores after regressing out age and gender. A clustering algorithm was applied to the patient data using performance on the choice reaction time task (based on reaction times and errors) in order to define a slow cognition patient group (n=170) and a non-slow cognition group (n=140). FIG. 11 shows tasks and measures for which there was a significant difference between slow cognition and non-slow cognition patients. As shown, slow cognition patients demonstrated poor cognition in the following measures: reduced verbal fluency (number of words produced in response to a cue); slowed reaction time in the simple reaction time task; slowed reaction time to congruent and incongruent stimuli, greater standard deviation of reaction times and increased errors in the Eriksen flanker task; prolonged Trails A and B time in the trail making test; reduced total correct trials in the digit symbol substitution test (DSST); shorter block span in the Corsi block task; greater total errors in the Wisconsin Card Sorting Task (WCST); reduced correct word recognition and reduced word recall after a distraction in the verbal learning and memory task; reduced accuracy of recognition of happy, angry or fearful faces in the face emotion recognition test; lower reward earned during the high reward and high win probability conditions, increased reward earned in the low win probability condition and increased hard task selection in the low win probability condition in the effortful expenditure for reward task; and slowed typing speed in the typing test. As such, these tasks and measures within them can be used to identify patients with poor or slow cognition.

### Example 2—Phase 1b Clinical Trial with NSI-189

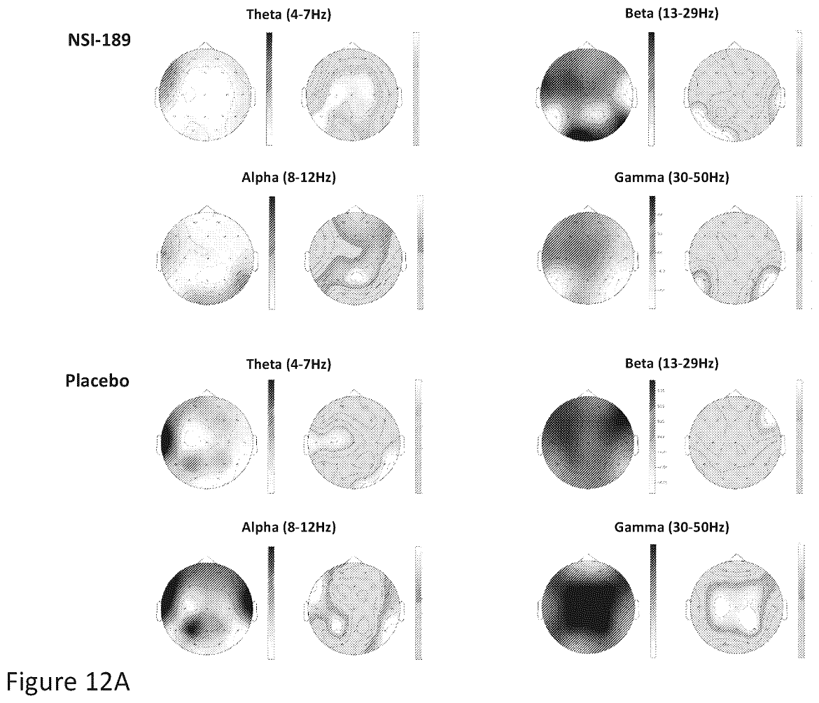

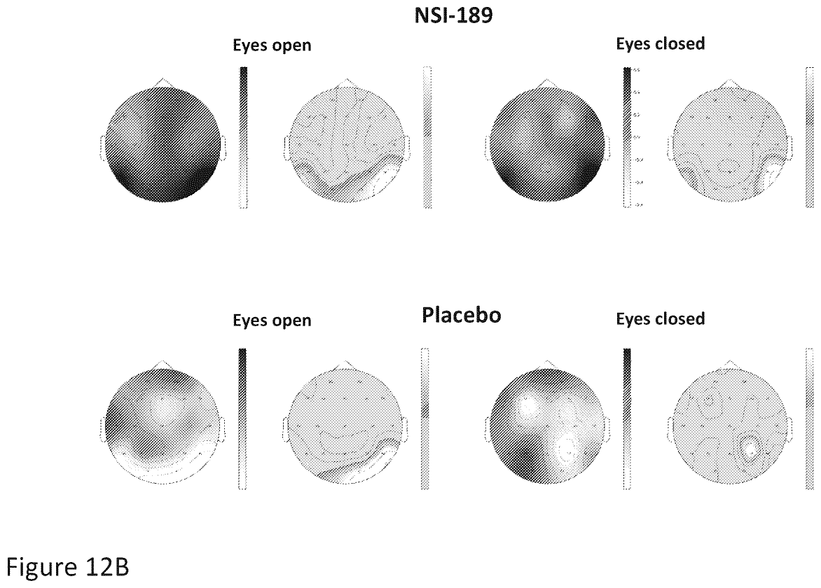

In addition to cognitive task behavior, whether brain activity, captured with electroencephalography (EEG) while patients rested with eyes open or eyes closed, could predict treatment outcome was examined. Such a finding would establish an additional modality by which patients who will benefit more from NSI-189 could be identified. To do so, resting EEG signals collected during the Phase 1b study on NSI-189, which included data on 18 patients who received one of three doses (total daily doses of 40 mg, 80 mg, or 120 mg) was examined, along with 6 patients who received placebo. In order to identify a drug predictive signal, the 18 patients on NSI-189 were pooled. Shown in FIG. 12A are EEG signals, plotted on a scalp topographic map, which significantly predicted treatment outcome, as measured by the change from baseline to end of treatment on the MADRS depression score. These EEG measures, which reflect channel-level frequency band-limited power, are one of multiple ways that resting brain activity can be quantified on EEG. Other common measures, which could likewise be used to identify future treatment responders, including various forms of connectivity (e.g. power envelope connectivity, coherence, power ratios between frequencies, cordance, imaginary coherence, phase locking value, phase lag index, weighted phase lag index, covariance, alpha peak frequency, alpha peak frequency proximity, and cross-frequency coupling) as well as other higher order measures such as information theoretical indices and entropy. As seen in FIG. 12A, lower power in parietal and frontal electrodes in the theta (4-7 Hz) and alpha (8-12 Hz) frequency ranges significantly predicted better treatment outcome with the drug but not placebo. Interestingly, theta frequency oscillations are an important means by which the hippocampus and prefrontal cortex communicate with each other (19). A reduced capacity for such communication may be related therefore to the therapeutic effects of NSI-189 due to their presumed neurogenic and pro-plasticity mechanisms, which have been observed in animal models and discussed above.

The power spectral density distribution of the EEG data was also examined. A schematic showing the EEG power spectral density is provided in FIGS. 1A and 1B in A. T. Hill et al., Dev. Cogn. Neurosci. 54:101076, 2022. The background trend in the data can be quantified as a measure of background neural activity and has a 1/f relationship (where f is frequency). Quantification of this signal is termed the aperiodic exponent, which is estimated as the negative slope for the line of best fit over the 1-50 Hz range of the power spectral density in the log-log space (T. Donoghue, et al., Nat Neurosci, 23 (12): 1655-1665 December 2020). As shown in FIG. 12B, higher aperiodic exponents in posterior electrodes (parietal, occipital, temporal) significantly predicted better treatment outcome with the drug but not placebo. Notably, recent studies showed that lower aperiodic exponents are mechanistically linked to increased excitatory relative to inhibitory neural activity (R. Gao, et al., e, 158:70-78, 2017). This finding suggests that NSI-189 may be suited for patients with impaired balance of excitatory and inhibitory neural activity, such as those with excessive inhibition relative to excitation.

### REFERENCES

1. Rodrigues R S, Paulo S L, Moreira J B, Tanqueiro S R, Sebastiao A M, Diogenes M J, Xapelli S. Adult Neural Stem Cells as Promising Targets in Psychiatric Disorders. Stem Cells Dev. 2020; 29:1099-1117.
2. Zarate C A, Jr., Singh J, Manji H K. Cellular plasticity cascades: targets for the development of novel therapeutics for bipolar disorder. Biol Psychiatry. 2006; 59:1006-1020.
3. Carli M, Aringhieri S, Kolachalam S, Longoni B, Grenno G, Rossi M, Gemignani A, Fornai F, Maggio R, Scarselli M. Is adult hippocampal neurogenesis really relevant for the treatment of psychiatric disorders? Curr Neuropharmacol. 2020.
4. Park S C. Neurogenesis and antidepressant action. Cell Tissue Res. 2019; 377:95-106.
5. Tunc-Ozcan E, Peng C Y, Zhu Y, Dunlop S R, Contractor A, Kessler J A. Activating newborn neurons suppresses depression and anxiety-like behaviors. Nat Commun. 2019; 10:3768.
6. Serafini G. Neuroplasticity and major depression, the role of modern antidepressant drugs. World J Psychiatry. 2012; 2:49-57.
7. Levy M J F, Boulle F, Steinbusch H W, van den Hove D L A, Kenis G, Lanfumey L. Neurotrophic factors and neuroplasticity pathways in the pathophysiology and treatment of depression. Psychopharmacology (Berl). 2018; 235:2195-2220.
8. Allen B D, Acharya M M, Lu C, Giedzinski E, Chmielewski N N, Quach D, Hefferan M, Johe K K, Limoli C L. Remediation of Radiation-Induced Cognitive Dysfunction through Oral Administration of the Neuroprotective Compound NSI-189. Radiat Res. 2018; 189:345-353.
9. Bauman M D, Schumann C M, Carlson E L, Taylor S L, Vazquez-Rosa E, Cintron-Perez C J, Shin M K, Williams N S, Pieper A A. Neuroprotective efficacy of P7C3 compounds in primate hippocampus. Transl Psychiatry. 2018; 8:202.
10. Fava M, Johe K, Ereshefsky L, Gertsik L G, English B A, Bilello J A, Thurmond L M, Johnstone J, Dickerson B C, Makris N, Hoeppner B B, Flynn M, Mischoulon D, Kinrys G, Freeman M P. A Phase 1B, randomized, double blind, placebo controlled, multiple-dose escalation study of NSI-189 phosphate, a neurogenic compound, in depressed patients. Mol Psychiatry. 2016; 21:1372-1380.
11. Papakostas G I, Johe K, Hand H, Drouillard A, Russo P, Kay G, Kashambwa R, Hoeppner B, Flynn M, Yeung A, Martinson M A, Fava M. A phase 2, double-blind, placebo-controlled study of NSI-189 phosphate, a neurogenic compound, among outpatients with major depressive disorder. Mol Psychiatry. 2020; 25:1569-1579.
12. Snyder H R. Major depressive disorder is associated with broad impairments on neuropsychological measures of executive function: a meta-analysis and review. Psychol Bull. 2013; 139:81-132.
13. Parkinson W L, Rehman Y, Rathbone M, Upadhye S. Performances on individual neurocognitive tests by people experiencing a current major depression episode: A systematic review and meta-analysis. J Affect Disord. 2020; 276:249-259.
14. Etkin A, Patenaude B, Song Y J, Usherwood T, Rekshan W, Schatzberg A F, Rush A J, Williams L M. A cognitive-emotional biomarker for predicting remission with antidepressant medications: a report from the iSPOT-D trial. Neuropsychopharmacology. 2015; 40:1332-1342.
15. Fava M, Iosifescu D V, Pedrelli P, Baer L. Reliability and validity of the Massachusetts general hospital cognitive and physical functioning questionnaire. Psychother Psychosom. 2009; 78:91-97.
16. Koenig A M, Bhalla R K, Butters M A. Cognitive functioning and late-life depression. J Int Neuropsychol Soc. 2014; 20:461-467.
17. Scott J C, Matt G E, Wrocklage K M, Crnich C, Jordan J, Southwick S M, Krystal J H, Schweinsburg B C. A quantitative meta-analysis of neurocognitive functioning in posttraumatic stress disorder. Psychol Bull. 2015; 141:105-140.
18. Hall M G, Hauson A O, Wollman S C, Allen K E, Connors E J, Stern M J, Kimmel C L, Stephan R A, Sarkissians S, Barlet B D, Grant I. Neuropsychological comparisons of cocaine versus methamphetamine users: A research synthesis and meta-analysis. Am J Drug Alcohol Abuse. 2018; 44:277-293.
19. Padilla-Coreano N, Canetta S, Mikofsky R M, Alway E, Passecker J, Myroshnychenko M V, Garcia-Garcia A L, Warren R, Teboul E, Blackman D R, Morton M P, Hupalo S, Tye K M, Kellendonk C, Kupferschmidt D A, Gordon J A. Hippocampal-Prefrontal Theta Transmission Regulates Avoidance Behavior. Neuron. 2019; 104:601-610 e604.
20. Egner T, Etkin A, Gale S, Hirsch J. Dissociable neural systems resolve conflict from emotional versus nonemotional distracters. Cereb Cortex 2008; 18:1475-84.
21. Xue S, Wang S, Kong X, Qiu J. Abnormal Neural Basis of Emotional Conflict Control in Treatment-Resistant Depression. Clin EEG Neurosci 2017; 48:103-10.
22. Xu M, Xu G, Yang Y. Neural Systems Underlying Emotional and Non-emotional Interference Processing: An ALE Meta-Analysis of Functional Neuroimaging Studies. Front Behav Neurosci 2016; 10:220.
23. Etkin A, Egner T, Peraza D M, Kandel E R, Hirsch J. Resolving emotional conflict: a role for the rostral anterior cingulate cortex in modulating activity in the amygdala. Neuron 2006; 51:871-82.
24. Maier M E, di Pellegrino G. Impaired conflict adaptation in an emotional task context following rostral anterior cingulate cortex lesions in humans. J Cogn Neurosci 2012; 24:2070-9.
25. Gyurak A, Patenaude B, Korgaonkar M S, Grieve S M, Williams L M, Etkin A. Frontoparietal Activation During Response Inhibition Predicts Remission to Antidepressants in Patients With Major Depression. Biol Psychiatry 2016; 79:274-81.
26. Williams L M, Korgaonkar M S, Song Y C, et al. Amygdala Reactivity to Emotional Faces in the Prediction of General and Medication-Specific Responses to Antidepressant Treatment in the Randomized iSPOT-D Trial. Neuropsychopharmacology 2015; 40:2398-408.
27. Johe et al., Ann Clin Psychiatry, 32 (3): 182-196 (2020)

All publications, patents and patent applications cited herein are hereby incorporated by reference as if set forth in their entirety herein. While this invention has been described with reference to illustrative embodiments, this description is not intended to be construed in a limiting sense. Various modifications and combinations of illustrative embodiments, as well as other embodiments of the invention, will be apparent to persons skilled in the art upon reference to the description. It is therefore intended that the appended claims encompass such modifications and enhancements.

1. A method of treating major depressive disorder in a human patient having objectively determined cognitive impairment or poor cognition comprising orally administering to the patient from about 40 to about 240 mg of (4-benzylpiperazin-1-yl)-[2-(3-methylbutylamino)pyridin-3-yl]methanone or a pharmaceutically acceptable salt thereof daily.

2. The method of claim 1, wherein about 60 to about 100 mg of (4-benzylpiperazin-1-yl)-[2-(3-methylbutylamino)pyridin-3-yl]methanone or a pharmaceutically acceptable salt thereof is administered daily.

3. The method of claim 1, wherein about 80 mg of (4-benzylpiperazin-1-yl)-[2-(3-methylbutylamino)pyridin-3-yl]methanone or a pharmaceutically acceptable salt thereof is administered daily.

4. The method of claim 1, wherein the human patient suffers from cognitive impairment, poor or slow cognition or difficulty making decisions as shown by one or more of a simple reaction time test, choice reaction time test, one back working memory task, and visual learning task.

5. The method of claim 4, wherein the human patient suffers from cognitive impairment, poor or slow cognition or difficulty making decisions as shown by a composite score which is at least partially based upon one or more results from a simple reaction time test, choice reaction time test, one back working memory task, or visual learning task.

6. The method of claim 1, wherein the patient suffers from anhedonia, suicidality, or both.

7. The method of claim 1, wherein the patient suffers from reduced information processing speed.

8. The method of claim 1, wherein the patient suffers from reduced attention, memory, learning, working memory, or any combination of any of the foregoing.

9. The method of claim 1, wherein the patient is not concurrently treated with a second antidepressant medication.

10. The method of claim 1, wherein the patient is concurrently treated with a second antidepressant medication.

11. A method of treating major depressive disorder in a human patient having reduced information processing speed, attention, memory, learning, working memory, or any combination of any of the foregoing, comprising orally administering to the patient from about 40 to about 240 mg of (4-benzylpiperazin-1-yl)-[2-(3-methylbutylamino)pyridin-3-yl]methanone or a pharmaceutically acceptable salt thereof daily.

12. The method of claim 11, wherein about 60 to about 100 mg of (4-benzylpiperazin-1-yl)-[2-(3-methylbutylamino)pyridin-3-yl]methanone or a pharmaceutically acceptable salt thereof is administered daily.

13. The method of claim 11, wherein about 80 mg of (4-benzylpiperazin-1-yl)-[2-(3-methylbutylamino)pyridin-3-yl]methanone or a pharmaceutically acceptable salt thereof is administered daily.

14-56. (canceled)

57. The method of claim 1, wherein the (4-benzylpiperazin-1-yl)-[2-(3-methylbutylamino)pyridin-3-yl]methanone or a pharmaceutically acceptable salt thereof is administered such that, at steady state the Cₘₐₓ for (4-benzylpiperazin-1-yl)-[2-(3-methylbutylamino)pyridin-3-yl]methanone is about 230 to about 630 ng/mL.

58. The method of claim 1, wherein the (4-benzylpiperazin-1-yl)-[2-(3-methylbutylamino)pyridin-3-yl]methanone or a pharmaceutically acceptable salt thereof is administered such that, at steady state the AUC₀₋₂₄ for (4-benzylpiperazin-1-yl)-[2-(3-methylbutylamino)pyridin-3-yl]methanone is about 1400 to about 3200 hr·ng/mL.

59. The method of claim 1, wherein the (4-benzylpiperazin-1-yl)-[2-(3-methylbutylamino)pyridin-3-yl]methanone or a pharmaceutically acceptable salt thereof is administered such that, at steady state the AUC₀₋ₜₐᵤ for (4-benzylpiperazin-1-yl)-[2-(3-methylbutylamino)pyridin-3-yl]methanone is about 1200 to about 2050 hr·ng/mL.

60. The method of claim 1, wherein the method comprises administering about 40 mg of (4-benzylpiperazin-1-yl)-[2-(3-methylbutylamino)pyridin-3-yl]methanone or a pharmaceutically acceptable salt thereof twice daily.

61. The method of claim 60, wherein the method comprises administering about 40 mg (4-benzylpiperazin-1-yl)-[2-(3-methylbutylamino)pyridin-3-yl]methanone phosphate twice daily.
